(19) United States
(12) Patent Application Publication
(10) Pub. No.: US 2024/0003890 A1
(43) Pub. Date: Jan. 4, 2024

(54) PARTICLE-BASED DETECTION OF ANALYTES

(71) Applicant: Xibus Syetems, Inc., Watertown, MA (US)

(72) Inventors: Timothy Manning Swager, Newton, MA (US); Kent Harvey, South Boston, MA (US); Matthias Oberli, Cambridge, MA (US)

(73) Assignee: Xibus Systems, Inc., Watertown, MA (US)

(21) Appl. No.: 18/037,279

(22) PCT Filed: Nov. 17, 2021
(86) PCT No.: PCT/US2021/059791

§ 371 Date: May 16, 2023

Related U.S. Application Data
(60) Provisional application No. 63/115,456, filed on Nov. 18, 2020.

(51) Int. Cl. G01N 33/58; G01N 33/543; G01N 33/545

(52) U.S. Cl. CPC G01N 33/582; G01N 33/54386; G01N 33/545

(57) ABSTRACT

Particle-based detection of analytes including, for example, systems, kits, and methods for growth, isolation, and/or monitoring of analytes are generally disclosed. In some embodiments, the systems and methods described herein are generally directed to the capture and/or concentrating of a target species (e.g., analyte) to be detected and/or monitored. In some embodiments, the materials, systems, and methods described herein may be used to create luminescent signals in response to the presence of selected analytes such as bacteria, viruses, and parasites. In some cases, the target analyte is a pathogenic bacteria, a pathogenic virus, a pathogenic parasite, or toxin.

### RELATED APPLICATIONS

This application claims priority under 35 U.S.C. § 119(e) to co-pending U.S. Provisional Application Ser. No. 63/115,456, filed Nov. 18, 2020, the contents of which are incorporated herein by reference in its entirety for all purposes.

### FIELD OF THE INVENTION

Embodiments described herein generally relate to particle-based detection of analytes and related methods, kits, and systems.

### BACKGROUND OF THE INVENTION

Rapid detection of pathogenic organisms, viruses, and other biomolecular targets (e.g., analytes) is central to ensuring food/water/beverage quality and to monitor and diagnose disease. In some applications it may be important to be able to determine if pathogenic organisms are alive or dead. It is also desirable, in some cases, to create methods that are capable of detecting the target species at trace levels.

For example, pathogenic bacteria in food and beverages represents a major health concern and can cause hospitalization and even death. Food security is of increasing importance and tainted products that cause sickness in the consumers is a major liability to producers and, beyond the consequences to human health, can cause economic damages in terms of brand erosion. Pathogenic bacteria include Listeria monocytogenes, E. coli, Salmonella enterica, Legionella, Campylobacter jejuni, and Toxoplasma gondii. Viruses can also spread through food including the influenza A, influenza B, and Norovirus. Improved methods are needed to rapidly detect these pathogens in food. In some cases, such monitoring and testing, especially for pathogenic and/or toxic analytes, may require the use of expensive and complex Bio safety Level 2 facilities or higher. Accordingly, improved systems and methods are needed.

### SUMMARY OF THE INVENTION

Systems, kits, and methods for growth, isolation, and/or monitoring of analyte (e.g., pathogenic analytes) are generally provided.

In one aspect, systems for monitoring of a pathogenic analyte are provided. In some embodiments, the system comprises a reservoir configured to receive a sample suspected of including the pathogenic analyte, wherein the reservoir is configured to be essentially closed with respect to the pathogenic analyte, a sterile growth medium formulated to preferentially grow the pathogenic analyte disposed within the reservoir, the growth medium comprising a plurality of isolation particles comprising a first moiety capable of binding to the pathogenic analyte, if present, and a plurality of signaling entities comprising a second moiety capable of binding to the pathogenic analyte, if present.

In some embodiments, the system comprises a reservoir configured to receive a sample suspected of comprising the pathogenic analyte, wherein the reservoir is configured to be essentially closed with respect to the pathogenic analyte once closed, a sterile growth medium formulated to preferentially grow the pathogenic analyte disposed within the reservoir, wherein the pathogenic analyte otherwise requires handling under BSL2 protocol, and wherein the system does not require a biological safety cabinet or other physical containment equipment to monitor the pathogenic analyte.

In some embodiments, the system comprises a vessel configured to contain a fluid, and including a localization and detection region, a source of a magnetic field configured to draw magnetic particles proximate the detection region, a source of excitation energy positioned to expose the detection region to the excitation energy, and a detector positioned to detect a signal emitted in the detection region.

In another aspect, kits are provided. In some embodiments, the kit comprises a reservoir configured to receive a pathogenic analyte and a growth medium, wherein the reservoir is configured to be essentially closed with respect to the pathogenic analyte once closed, a sterile growth medium formulated to preferentially grow the pathogenic analyte disposed within the reservoir, the growth medium comprising a plurality of isolation particles comprising a first moiety capable of binding to the pathogenic analyte.

In yet another aspect, methods for monitoring growth of a pathogenic analyte are provided. In some embodiments, the method comprises introducing a sample suspected of comprising the pathogenic analyte into a reservoir, introducing a sterile growth medium formulated to preferentially grow the pathogenic analyte into the reservoir, closing the reservoir with respect to the pathogenic analyte, culturing, for a desired period of time, the sample, mixing the growth medium comprising the sample with a plurality of isolation particles comprising a first moiety capable of binding to the pathogenic analyte, if present, isolating, via the plurality of isolation particles, the pathogenic analyte, and determining a property of the pathogenic analyte.

In some embodiments, the method comprises exposing a medium suspected of containing an analyte to a set of magnetic particles and a set of non-magnetic signaling entities, and, if the analyte is present, allowing the analyte to link at least some of the magnetic particles with at least some of the non-magnetic signaling entities, localizing at least some of the magnetic particles linked to non-magnetic signaling entities via the analyte proximate a source of excitation energy, and localizing at least some of the magnetic particles linked to non-magnetic signaling entities via the analyte proximate a detector, and exciting signaling entities with the source of excitation energy, and determining a signal via the detector, thereby determining a characteristic of the analyte.

In another aspect, compositions are provided. In some embodiments, the composition comprises a conjugated polymeric species in the form of a signaling entity, optionally, a particulate signaling entity.

Other advantages and novel features of the present invention will become apparent from the following detailed description of various non-limiting embodiments of the invention when considered in conjunction with the accompanying figures. In cases where the present specification and a document Incorporated by reference include conflicting and/or inconsistent disclosure, the present specification shall control

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A is an schematic diagram of an exemplary system for monitoring an analyte, according to one set of embodiments.

FIG. 1B is an illustration of the measurement with a fiber optic probe of the emission (e.g., luminescence at 615 nm) from a solution that has non-magnetic luminescent particles suspended in a solution, according to one set of embodiments. The particles were observed to drop to the bottom of the vessel over time and the fluorescent signal decreases from 1 to 2 to 3 as shown in photos and schematically.

FIG. 2 shows plots at the top which represent measurements on three separate vials filled with different sized droplets (65, 35, and 20-30 microns, shown at the bottom) of the fluid shown dispersed in water with surfactants, according to one set of embodiments. In some embodiments, the droplets contain an emissive dye and the plots correspond to a fluorescence signal of the solution obtained with a bifurcated fiber optic probe mid-way up the vial. When the droplets are suspended in the water by agitation fluorescence is observed, which decays upon sitting as the droplets sink the bottom of the vial. For example, in some embodiments, the larger droplets sink with in one minute, the medium droplets take 4-5 minutes, and smallest droplets take approximately 9 minutes to settle on the bottom.

FIG. 3 is a schematic illustration of how magnetic and non-magnetic particles functionalized with receptors for target analytes may become associated by binding to a multivalent analyte, according to one set of embodiments. The non-limiting example of the receptor is a rcSso7d engineered protein is illustrated and the signaling entity is an emissive dye. Other analytes, receptors (e.g., moieties), signaling entities, are also possible. The magnetic particles may also contain signaling entity, in some cases.

FIG. 4 is a schematic illustration of an exemplary detection of a biological analyte by magnetic localization at the sidewall of a vessel, according to one set of embodiments. For example, a magnet is colocalized with a bifurcated fiber optic capable of exciting a sample and collecting luminescence or reflected light. In the case that the analyte is present (top) the analyte may produce, in some embodiments, a connection between the magnetic beads and the non-magnetic luminescent or reflective beads.

Capture of the magnetic particles at the location may generally allow for the recording of a signal (optical output). If the biological analyte is not present then, for example, the non-magnetic particles will not be co-localized with the magnetic beads. The magnetic beads may be optically encoded separately with an emissive dye to provide for quantitation of the biological analyte and to confirm that the beads are localized at the desired location in the absence of the biological analyte, in some embodiments.

FIG. 5 is a schematic illustration of an exemplary flow channel and the localization of particles and analyte by a magnet, according to one set of embodiments. In some embodiments, the fiber optical is places the other side of the flow channel and the light may be collected and focused into the fiber optic by a lens in the optical path.

FIG. 6 is a schematic illustration of two exemplary configurations for determination of an analyte in a plastic bag containing analyte, according to one set of embodiments. The exemplary schematics show the localization of the magnetic particles and nonmagnetic particles in response to the presence of analyte.

### DETAILED DESCRIPTION

Particle-based detection of analytes including, for example, systems, kits, and methods for growth, isolation, and/or monitoring of analytes are generally disclosed. In some embodiments, the systems and methods described herein are generally directed to the capture and/or concentrating of a target species (e.g., analyte) to be detected and/or monitored. In some embodiments, the materials, systems, and methods described herein may be used to create luminescent signals in response to the presence of selected analytes such as bacteria, viruses, and parasites. In some embodiments, the target analyte is a pathogenic bacteria, a pathogenic virus, a pathogenic parasite, or toxin. In some embodiments, the analyte is a cell. In some embodiments, the analyte comprises a protein, a toxin, RNA, DNA, an antibody, or combinations thereof.

The phrase “pathogenic analyte” and “pathogens” as used herein is given its ordinary meaning in the art and generally refers to an analyte (e.g., bacteria, virus, parasite, fungus) that causes disease in a subject.

A “subject” refers to any animal such as a mammal (e.g., a human). Non-limiting examples of subjects include a human, a non-human primate, a cow, a horse, a pig, a sheep, a goat, a dog, a cat or a rodent such as a mouse, a rat, a hamster, a bird, a fish, or a guinea pig. Generally, the invention is directed toward use with pathogenic analytes that may cause disease in humans.

The systems and methods described herein may have numerous advantages over traditional methods for growing, isolating, and/or monitoring analytes of interest such as pathogens. For example, the systems and methods disclosed herein may, in some embodiments, have advantages in terms of simplicity, efficiency, and safety when analyzing for pathogenic or toxic biological species. In some embodiments, the systems described herein comprise a reservoir (e.g., container, vessel, channel, bag) that is advantageously configured to be essentially closed to the analyte. That is to say, in some embodiments, the analyte, once placed within the reservoir, is essentially unable to be removed from the reservoir (except for the physical and/or mechanical breakage of the reservoir). Advantageously, systems which incorporate reservoirs as described herein may permit the analysis, monitoring, and growth of pathogenic analytes without the use of expensive and complex safety equipment and/or Biosafety Level 2 (BSL2) or higher protocols and facilities. That is to say, in some embodiments, the systems and methods described do not require a biological safety cabinet or other physical containment equipment to monitor the pathogenic analyte.

In some embodiments, the use of the systems and methods described herein may advantageously provide important information about the analyte including whether the analyte is alive or dead. Advantageously, methods described are highly sensitive capable of detecting less than 100 colony forming units (CFUs) per mL rapidly, are low cost, and/or may be used with devices that enable many tests to be conducted simultaneously.

There are generally a number of possible arrangements for detection and/or determination of analytes described in this specification. In an exemplary set of embodiments, the method comprises exposing an analyte to a plurality of isolation particles (e.g., magnetic particles and/or other particles capable of being manipulated and/or moved by an external field or force) and a plurality of signaling entities (e.g., non-magnetic particles comprising a fluorescent moiety, fluorescent particles). In some embodiments, the plurality of isolation particles and/or the plurality of signaling entities comprise a moiety capable of binding to the analyte. In some embodiments, the plurality of isolation particles comprise a first moiety capable of binding to the analyte. In some embodiments, the plurality of signaling entities comprise a second moiety capable of binding to the analyte. In some embodiments, the first moiety and the second moiety are the same and/or are configured to bind to the same portion of the analyte. In some embodiments, the first moiety and the second moiety are different and/or are configured to bind to different portions of the analyte.

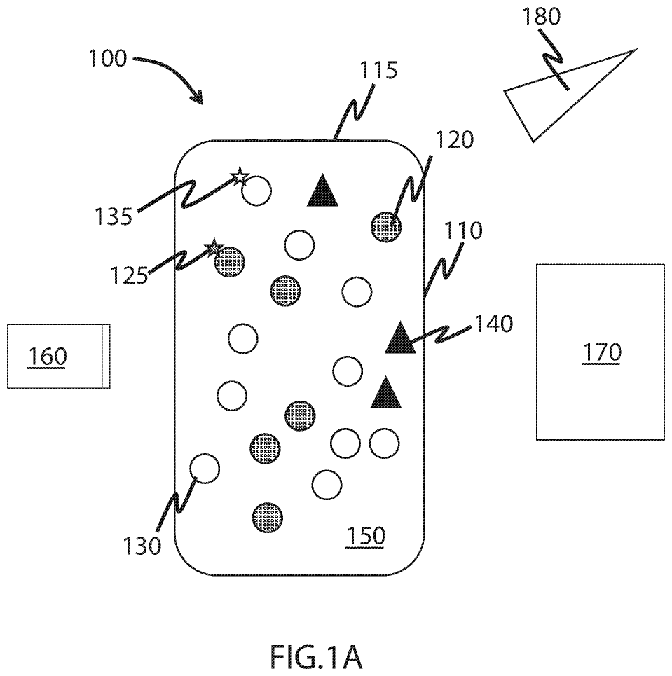

For example, as illustrated in FIG. 1A, system 100 comprises a reservoir 110. In some embodiments, reservoir 110 is configured to receive a sample (e.g., via opening 115) suspected of including a pathogenic analyte (e.g., analyte 140). In some embodiments, reservoir 110 may be essentially closed with respect to the analyte (e.g., the pathogenic analyte). For example, in some cases, once the sample is placed within reservoir 110, the analyte is unable to exit reservoir 110 (absent, for example, physical or mechanical breakage of reservoir 110). Non-limiting examples of suitable reservoirs include containers, vials, vessels, fluidic channels (e.g., including microfluidic channels of a microfluidic device), sample bags, or the like. Those of ordinary skill in the art would be capable of selecting, based upon the teachings of this specification, addition suitable reservoirs which may be essentially closed with respect to the analyte, as described herein.

In some embodiments, reservoir 119 is essentially closed with respect to the analyte while opening 115 permits addition of one or more materials (e.g., fluids, reagents, particles, entities, or the like) while preventing the passage of the analyte. The opening may be in fluidic communication with one or more valves, pipes, tubes, inlets, outlets, or other fluidic components to permit the addition and/or removal of various materials (e.g., fluids, reagents, particles, entities, or the like). In some embodiments, the opening comprises a selective membrane and/or filter which prevents the passage of the analyte but permits the passage of one or more materials (fluids, reagents, particles, entities, or the like) that do not comprise the analyte.

In an exemplary set of embodiments, environmental samples collected from surfaces, from water washes, or directly from food are transferred to a detection-growth reservoir, as described herein. This reservoir may be closed, in some embodiments, in the sense that bacteria are unable to escape this system and are completely contained. The ability of the reservoir to be isolated is also useful to maintain a pre-sterilized growth media and the detection particle mixtures. In some cases, if sterility is not maintained, other bacterial can begin to grow, for example, in the reservoir prior to use. A key advantage of the isolation of samples in a detection-growth reservoir is that it will allow for testing in facilities that lack biological safety laboratories (BSL). Generally, to handle live pathogenic bacteria that are not in a sealed system a BSL-2 facility is required. As such, detection and growth containers that effectively maintain this containment may allow, in some cases, for expanded testing for food pathogens in facilities that do not have BSL-2 capabilities. Traditional pathogen detection is generally not performed at many small food production facilities for lack of the BSL-2 capabilities, and samples are usually sent out to other providers. Additionally, the lack of BSL-2 facilities often prevents food distributors from testing on site. Advantageously and illustratively, using the systems and methods disclosed herein, farmers could test for pathogenic organisms on site. The closed systems described herein provide the necessary safety for pathogen detection at sites including, for example, retail stores and restaurants, and may thereby enable broader pathogen testing than was previously possible. An expanded testing is in the public interest and will prevent sickness and deaths that occur from pathogen testing.

The detection-growth reservoir may have many different form factors and may be a flexible plastic bag, a rigid hard plastic, metal, or glass container, or combinations thereof, in addition to the examples disclosed herein. In some embodiments, the reservoir may also be fitted with equipment that allows for other materials to be injected. For example, it could have valves (e.g., associated with one or more openings such as opening 115 in FIG. 1A) that allow for material to pumped into the reservoir. Alternatively, the reservoir may comprise a septum that allows for the injection of materials through a syringe needle or the like. Bacteria samples of interest added to these detection-growth reservoirs will generally multiply and the magnetic localization methods allow for periodic measurement of the optical signals as an indication of presence of bacteria. As the bacteria grow in the reservoir, they become dangerous if released and hence a core advantage of the use of the detection and growth reservoir is that the bacteria generally never leave the reservoir as viable living organisms (e.g., except in the case of unwanted breakage of the reservoir). After monitoring the bacteria by one of more measurements over time and making a determination about the quantity of viable bacteria, the closed system may be sterilized by the addition of chemical reagents (peroxides, bleach, etc.), intense UV treatment, or thermal treatments. In some embodiments, the reservoirs are reused and in other cases the reservoirs are designed for a single use. Sterilized devices may be discarded. In some embodiments the sterilization and disposal may be accomplished by incineration of the device. In the latter, the materials may be chosen to make this process as environmentally friendly as possible.

As described herein, the detection-growth reservoir may be initially charged with a sterile media that is selected to promote the growth of selected bacteria and optionally to suppress the growth of other bacteria. It is also possible that this mixture may be fortified over time by injections of additional media or gas. In some embodiments the detection-growth reservoir is completely hermetically sealed. No gas, liquid or other matter is introduced or withdrawn after sample introduction until after the camber is sterilized or destroyed, in some embodiments. In other embodiments, the reservoir is fitted with a semipermeable membrane that will allow for the transfer of gas or liquids, but will not allow bacteria to pass. These filters may have small pore sizes of 0.2 microns or have hydrophobic characteristics that prevent bacteria transport. In some embodiments the ability of gas or solutions to be added or withdrawn from the detection-growth reservoir may assist in the detection of bacteria. This could include simply concentrating the bacteria, but could also involve adding gas or nutrients that enhance the growth of the target bacteria and/or suppress the growth of others. For example, if the atmosphere is kept as oxygen free, only anerobic bacteria (c.a. Campylobacter) are expected to grow.

It is also possible that the detection-growth system may have multiple reservoirs, in some cases. In an exemplary embodiment, the sample is initially inserted into a reservoir and isolated. This reservoir may have a solution that extracts the bacteria and then distributes the extract into one or more other reservoirs that contain growth media that is designed to promote the growth of one target bacteria over the others. The one or more other reservoirs may also contain particles for magnetic localization and optical signaling that are specific to an analyte of class of analytes. In this way a single sample may be analyzed for multiple bacteria and/or other analytes. The distribution to the multiple isolated detection-growth reservoirs could be done in parallel or in series. In the latter, in some embodiments the sample can be first grown in one medium that promotes the growth a specific pathogen and then a portion of that growth mixture can be transferred to another reservoir that favors the growth of one or more other pathogens.

In some embodiments, once the sample is collected and placed in the reservoir the reservoir does not comprise an opening. In some embodiments, the reservoir is effectively sealed (e.g., hermetically sealed) such that all added materials including, for example, one or more of the sample suspected of including the analyte, the isolation particles, the signaling entities, growth media, etc. are not readily removable from the reservoir (e.g., absent physical or mechanical breakage of the reservoir).

In some embodiments, a plurality of isolation particles 120 (e.g., magnetic particles) may be added to reservoir 110. In some embodiments, the plurality of isolation particles are added to the sample prior to adding the sample to the reservoir. In some embodiments, the plurality of isolation particles are added directly to the reservoir (e.g., before adding the sample, after adding the sample).

In some embodiments, plurality of isolation particles 120 are configured to bind to analyte 140, if present. In some embodiments, plurality of isolation particles 120 comprise a moiety 125 capable of binding with analyte 140, if present.

In an exemplary set of embodiments, the isolation particles comprise magnetic particles or any other particle generally capable of being manipulation and/or moved (e.g., via an external force and/or field). A wide variety of isolation particles (e.g., magnetic particles) may be used in connection with the invention. Those of ordinary skill in the art would be capable of selecting magnetic particles able to be functionalized with linking or binding moieties for chemical, biological, or biochemical analysis, and any such particles may be utilized with the invention. In some embodiments, the plurality of isolation particles further comprise a detectable emissive species. Additional suitable isolation particles are described, below.

In some embodiments, a plurality of signaling entities 130 (e.g., fluorescent particles) may, optionally, be added to reservoir 110. In some embodiments, the plurality of signaling entities are added to the sample prior to adding the sample to the reservoir. In some embodiments, the plurality of signaling entities are added directly to the reservoir (e.g., before adding the sample, after adding the sample).

In some embodiments, plurality of signaling entities 130 are configured to bind to analyte 140, if present. In some embodiments, plurality of signaling entities 130 comprise a moiety 135 capable of binding with analyte 140, if present.

Signaling entities are known in the art, and those of ordinary skill in the art would be capable of selecting suitable signaling entities to be used in the invention based upon the teachings of this specification. In a subset of embodiments, signaling entities described below are used. Signaling entities may be particulate or non-particulate. Where non-particulate, they may be chemical (small molecule or polymers) or biological species. Where particulate, in one set of embodiments they are non-magnetic so that they are not brought proximate an excitation/detection region unless carried there by a magnetic particle to which they are linked optionally via an analyte. Particulate signaling entities may define particles which themselves may emit detectable signals through emission or the like, or may be functionalized on one or more surfaces with such signaling entities. For example, a signaling particle may have a surface or interior functionalized with signaling entities which signal when exposed to a source of excitation energy described herein, and may also be functionalized with linking moieties to link to an analyte which in turn may link a magnetic particle.

In some embodiments, the signaling entity is a non-magnetic particle. In some embodiments, the signaling entity is a recognition element (i.e. moiety) that is emissive. In some embodiments, the signaling entity is an emissive particle containing one or more recognition elements. In some embodiments, the signaling entity is an emissive polymer containing one or more recognition elements. In some embodiments, the signaling entity contains an emissive species containing Eu, Tb, Gd, Au, Au, Jr, Cu, Pd, Pt, Ru, Ag, Zn, or Al. In some embodiments, the signaling entity is an emissive polymer is a polyfluorene containing one or more recognition elements. In some embodiments, the signaling entity is an emissive polymer capable of transferring energy to a minority chromophore that one or more recognition elements.

In an exemplary set of embodiments, the plurality of signaling entities comprise a receptor-dye conjugate (e.g., comprising an antibody or recognition protein) and/or a nanoparticle (e.g., an emissive nanoparticle). Other signaling entities are also possible and are described in more detail, below. In some embodiments, the signaling entity is selected to have an excited state lifetime of greater than or equal to 1 microsecond (e.g., greater than or equal to 2 microseconds, greater than or equal to 5 microseconds, greater than or equal to 10 microseconds). In some embodiments that signaling entity is selected to have an excited state lifetime less than 1 microsecond, less than 100 nanoseconds, or less than 10 nanoseconds. In some embodiments, the signaling entity is configured to scatter light.

In some embodiments, a moiety (e.g., moiety 125, moiety 135) interacts with the analyte (e.g., pathogenic analyte) via formation of a bond, such as an ionic bond, a covalent bond, a hydrogen bond, Van der Waals interactions, and the like. The covalent bond may be, for example, carbon-carbon, carbon-oxygen, oxygen-silicon, sulfur-sulfur, carbon-sulfur, phosphorus-nitrogen, carbon-nitrogen, metal-oxygen, or other covalent bonds. The hydrogen bond may be, for example, between hydroxyl, amine, carboxyl, thiol, and/or similar functional groups. For example, the moiety may include a functional group, such as a thiol, aldehyde, ester, carboxylic acid, hydroxyl, and the like, wherein the functional group forms a bond with (a portion of) the analyte. In some cases, the moiety may be an electron-rich or electron-poor moiety wherein interaction between the moiety and the analyte comprises an electrostatic interaction.

Those of ordinary skill in the arts can select not only magnetic particles and signaling entities, but also linkages that can find link a magnetic particle two and analyze, and the analyte to a signaling entity. Any such linkages can be used in the invention or, in a subset of embodiments, linkages described below can be used. Linking moieties useful for linking magnetic particles to analyze and analyze two signaling particles or entities are known and any can be used in the invention. In a subset of embodiments, linking moieties described below can be used. “Link,” or “linking,” In connection with his invention means holding two entities linked (e.g., a magnetic particle and an analyte, or an analyte and a signaling entity), Directly or indirectly, such that the entities linked will move together such that if one is within an excitation region or detection region, the other will be within that region at least 70%, 80%, or 90% of the time. For example, where a magnetic particle is linked to an analyte which is linked to a signaling entity, and the magnetic particle is localized proximate a source of excitation energy and a detector, regardless of how the linkages are facilitated the signaling entity will respond to excitation and produce a detectable signal sufficiently such that analytes may be determined. Linkages may be and typically are organic moieties with specific binding partners or recognition elements as are known in the art.

In some cases, the moiety may comprise a biological or a chemical group capable of binding another biological or chemical molecule. For example, the moiety may include a functional group, such as a thiol, aldehyde, ester, carboxylic acid, hydroxyl, and the like, wherein the functional group forms a bond with (a portion of) the analyte.

In some embodiments, moiety and the analyte interact via a binding event between pairs of biological molecules including proteins, nucleic acids, glycoproteins, carbohydrates, hormones, and the like. Specific examples include an antibody/peptide pair, an antibody/antigen pair, an antibody fragment/antigen pair, an antibody/antigen fragment pair, an antibody fragment/antigen fragment pair, an antibody/hapten pair, an enzyme/substrate pair, an enzyme/inhibitor pair, an enzyme/cofactor pair, a protein/substrate pair, a nucleic acid/nucleic acid pair, a protein/nucleic acid pair, a peptide/peptide pair, a protein/protein pair, a small molecule/protein pair, a glutathione/GST pair, an anti-GFP/GFP fusion protein pair, a Myc/Max pair, a maltose/maltose binding protein pair, a carbohydrate/protein pair, a carbohydrate derivative/protein pair, a metal binding tag/metal/chelate, a peptide tag/metal ion-metal chelate pair, a peptide/NTA pair, a lectin/carbohydrate pair, a receptor/hormone pair, a receptor/effector pair, a complementary nucleic acid/nucleic acid pair, a ligand/cell surface receptor pair, a virus/ligand pair, a Protein A/antibody pair, a Protein G/antibody pair, a Protein L/antibody pair, an Fc receptor/antibody pair, a biotin/avidin pair, a biotin/streptavidin pair, a drug/target pair, a zinc finger/nucleic acid pair, a small molecule/peptide pair, a small molecule/protein pair, a small molecule/target pair, a carbohydrate/protein pair such as maltose/MBP (maltose binding protein), a small molecule/target pair, or a metal ion/chelating agent pair. Specific non-limiting examples of moieties include peptides, proteins, DNA, RNA, PNA. Other moieties and binding pairs are also possible.

Referring again to FIG. 1A, in some embodiments, a magnetic field source 160 (e.g., a magnet, a source of electric current) may be placed proximate reservoir 110. In some embodiments, the magnetic field source generates a magnetic field such that the plurality of isolation particles are drawn towards the magnetic field source. In some embodiments, an analyte that is bound to the plurality of isolation particles will similarly be drawn towards the magnetic field source.

In some embodiments, a detector (e.g., a fiber optic cable, a sensor) 170 capable of detecting electromagnetic radiation is positioned proximate reservoir 110. In some embodiments, a source of electromagnetic radiation 180 is positioned proximate the reservoir (e.g., to provide an excitation that causes the signaling entities to generate detectable electromagnetic radiation).

In an exemplary set of embodiments, a property of an analyte may be determined by activating the magnetic field source such that the isolation particles bound to the analyte are drawn towards the detector, and the detector is configured to detect a signal from the plurality of signaling entities (e.g., also bound to the analyte), optionally in the presence of applied electromagnetic radiation. In some embodiments, the signal may be correlated with the property of the analyte.

In some embodiments, the property of the analyte includes viability (e.g., alive, dead), presence of the analyte in the sample, a growth rate of the analyte, and/or a relative amount of analyte. In an exemplary set of embodiments, repeated measurements and the use of specific receptors enable the determination if bacteria are alive or dead.

In another exemplary set of embodiments, a plurality of magnetic particles and a plurality of non-magnetic signaling entities are added to a sample suspected of including an analyte. If analyte is present, then it may bind to at least one magnetic particle and at least one signaling entity so that when the magnetic particle is localized, via a magnetic field, at an excitation and/or detection region (e.g., which are the same, in one set of embodiments), excitation of the signaling entity and detection of a signal signifies presence of the analyte which, if it were not present, would result in the magnetic particle without signaling entity being drawn to the excitation/detection region. It is a feature of one set of embodiments that magnetic particle/analyte/signaling entity groups are localized at a region where the signaling entity may be excited and detected. In one set of embodiments, also described below, a ratiometric determination is made that may provide greater accuracy in detection by reducing or normalizing background/noise signaling. In this arrangement, a set of reference magnetic particles may be linked to reference signaling entities regardless of the presence or absence of any analyte, so when they are localized at an excitation/detection region, they provide a baseline signal against which the anolyte determination signal may be calibrated when analyte is introduced to the system.

In some embodiments, the optical excitation produces an emission that can be captured as an image and analyzed. This analysis may, in some cases, include the shape, size, intensity, and color of the emission image. This image data may be used to discern signals from non-specific binding of signaling groups to non-analyte particles and thereby eliminate false positive signals.

In one set of embodiments, the magnetic particles are localized proximate a source of excitation energy, and localized proximate a detector, and those regions are different. In this case the regions may be overlapping, but need not be. In another set of embodiments, those regions are essentially the same. Where they are the same, the invention may involve localizing the particles proximate both the source of excitation energy and the detector which, in this embodiment, may be referred to as an excitation/detection region. “Proximate,” in this context, means a sufficient quantity of magnetic particles are positioned in relation to the source of excitation energy such that a sufficient number of signaling entities linked to such particles may be excited so as to allow a detector to determine a signal indicative of the presence and/or quantity of signaling entity in the detection region (or, where the detection region and excitation region are the same, the excitation/detection region; at all locations herein, where an excitation region or region proximate a source of excitation is described, or a detection region or region proximate a detector is described, it is to be understood that these regions be essentially the same and this may be an excitation/detection region. It is also to be understood that where an excitation/detection region is described, in other embodiments the excitation region may be separate from the detection region, or the excitation region and detection region may be merely overlapping to some extent.

In another set of embodiments, the excitation region and detection region are essentially the same. As used in this context, “essentially the same” means when a magnetic field is applied to localize a set of magnetic particles linked to signaling entities at a location, and the magnetic field remains essentially unchanged such that the localization of the magnetic particles remains essentially unchanged, and excitation energy is applied to the excitation region, the detector may measure at least 20%, 40%, 60%, 80%, or 90% of emission from the signaling entities excited by such excitation energy.

In some embodiments, a growth medium (e.g., a sterile grown medium) 150 may be added to reservoir 110. In some embodiments, the growth medium is selected to preferentially grow the analyte of interest (e.g., the pathogenic analyte) disposed within the reservoir. One of ordinary skill in the art would be capable of selecting, based upon the teachings of this specification, growth medium which may preferentially grow an analyte of interest such as a pathogenic analyte. In some embodiments, the growth medium may permit the growth of the analyte of interest while inhibiting, killing, or otherwise growing at a substantively slower rate, other analytes such as other pathogenic analytes (e.g., pathogenic bacteria, pathogenic virus) and/or non-pathogenic analytes (e.g., non-harmful bacteria) that are not of interest.

In an illustrative embodiment, the (sterile) growth medium permits the growth of a pathogenic bacteria of interest while no other pathogenic bacteria and non-pathogenic bacteria is permitted to grow, such that, over time, the pathogenic bacteria of interest becomes the dominant species in the sample. Advantageously, the use of such selective growth medium may permit the long-term monitoring of pathogenic bacterial growth. In some embodiments, the systems and methods described herein may be used to determine if the pathogenic bacteria present in the sample is alive or dead.

The term “alive” generally refers to an analyte (e.g., bacteria) that, given optimal conditions in which the analyte (e.g., bacteria) is expected to grow, the analyte (e.g., bacteria) will multiply. In some cases, depending on the state of the analyte when collected in a sample, this process may be delayed while the analyte in effect heal over the course of a few hours. By way of example and for illustrative purposes only, if the pathogenic bacteria does not grow in the growth medium that otherwise preferentially permits the growth of the pathogenic bacteria, the pathogenic bacteria may be considered non-viable or dead.

Non-limiting examples of pathogenic analytes with which the systems and methods may be used to monitor, grow, and/or detect the presence of include Achromobacter xylosoxidans, Acinetobacter baumannii, Actinomyces (e.g., Actinomyces israelii), Aeromonas species, Bacillus species, Bacteroides fragilis, Bacteroides melaninogenicus, Bartonella species, Bordetella pertussis, Borrelia species, Brucella species, Burkholderia species, Campylobacter, Capnocytophaga species, Chlamydophila pneumoniae, Chlamydophila psittaci, Citrobacter species, Clostridium species, Corynebacterium species, Coxiella burnetiid, Ehrlichia species, Eikenella corrodens, Enterobacter species, Enterococcus faecalis, Enterococcus faecium, Escherichia coli, Francisella tularensis, Fusobacterium necrophorum, Gardnerella vaginalis, Haemophilus species, Helicobacter Pylori, Influenza (e.g., influenza A, influenza B), Klebsiella species, Lactobacillus species, Legionella species, Leptospira species, Listeria monocytogenes, Moraxella, catarrhalis, Morganella species, Mycoplasma pneumonia, Neisseria species, Nocardia species, Norovirus, Pasteurella multocida, Peptostreptococcus species, Porphyromonas gingivalis, Propionibacterium acnes, Proteus species, Providencia species, Pseudomonas aeruginosa, Salmonella species, Serratia marcescens, Shigella species, Staph epidermidis, Staph hominis, Staph. Haemolyticus, Staphylococcus aureus, Staphylococcus saprophyticus, Stenotrophomonas maltophilia, Streptococcus agalactiae, Streptococcus anginosus group, Streptococcus pneumoniae, Streptococcus pyogenes (Groups A, B, C, G, F), Toxoplasma gondii, Treponema pallidum, and Vibrio species.

In an exemplary set of embodiments, the analyte of interest may be selected from the group consisting of Listeria monocytogenes, E. coli, Salmonella enterica, Legionella, Campylobacter jejuni, Toxoplasma gondii, influenza A, influenza B, and Norovirus. Other analytes are also possible and those of ordinary skill in the art would be capable of selecting suitable target analytes based upon the teachings of this specification.

The systems and methods described herein may be used in a wide variety of applications in which detection of pathogenic analytes are important. For example, detecting organisms in water is also of interest and, in some embodiments, it may be ideal to detect classes or bacteria rather than individual bacteria. In this way total bacterial counts can be deduced. Total bacterial counts may use recognition elements (e.g., moiety) able to bind to a wide range of species.

In some embodiments, detecting and tracking of the outbreak of human disease using efficient detection methods of viruses is important to public health. The methods in this disclosure are also relevant to the detection of viruses and antibodies that can inform if an individual has previously contracted a disease.

Sensing of biological species generally uses mechanisms for their selective recognition and the ability to generate a readable signal associated with the recognition process. In general, an optical excitation is applied to a material and an emissive signal may be detected. Emissive represents all photons that are the result of luminescence, scattering, or reflection. The wavelength or color of the emission may also contain information and luminescence will generate selected ranges of light. Scattered light may have different ranges of wavelengths as a result of the particle size of because of subtractive color. Reflectivity may similarly have different intensities as a function of wavelength. In the case of a reflection that angle of the reflective surface of periodic structure with regard to the direction of the excitation and direction at which the light is collected will influence the wavelength intensity distribution of the emissive signal.

There are multiple ways to generate optical signals using luminescent probes have been widely used to study biological systems. Methods that create robust detection schemes capable of automation or with minimal user training may also benefit from the use of luminescent methods. An advantage of a luminescent method is that it may provide high sensitivity, if background signals may be eliminated. Background signals may include native fluorescence from the biological materials, media, containers, contaminates, or from stray light. There are different approaches to reduce or eliminate background that may include using filters to prevent the excitation of emissive species, or to prevent the light from unwanted emissions to reach the photodetectors. Delayed luminescent probes and time-gated emission detection may also be used to collect signals from particular probes at times after background emission has subsided.

Localization of emissive species in a place wherein they would not be present without a biological signal is described in this disclosure as an additional method for the creation of detectable emission signals and low background in the absence of the biological target. Specifically, by creating a measurement method wherein emissive species will only persistently associate at a location in the presence of a biological target signal may be used to produce a robust and sensitive signal. Optimal systems optimally making these types of measurements will take into consideration methods to reduce or eliminate scattered, stray light, and interference from other emissive sources. Hence, filters, directional detection, waveguiding, and focusing optics may be used to produce a variety of systems suitable for different applications. In some embodiments the measurement may be made inside of a sample bag. For example, in some cases a sample to be analyzed will be placed inside of a bag that will be mechanically stimulated to affect the release of bacteria. The sample may be food, tissue, plants, or a device such as a sponge that was used to collect bacterial from surfaces. Devices that provide this mechanical stimulation include what are known as stomachers and may be used for analysis for bacterial associated with food production and quality control. If the biological target analyte may cause localization of emissive species at a predetermined location, detection may be enabled without transferring the solution to another vessel. Such a process has advantages in terms of simplicity, efficiency, and safety when analyzing for pathogenic or toxic biological species. In other cases, it may be advantageous to transfer material from a bag after mechanical stimulation to liberate biological target analytes, into one or more additional vessels for analysis. The measurement within this new vessel may also make use of the ability the biological analyte to localize an emissive species at a location for measurement. Multiple vessels may be used to create specific tests for different species or to optimally extract information such as if pathogenic organisms are alive or dead, as well as provide different dynamic ranges of sensitivity. For example, vessels may be configured to provide ultralow limits of detection in a very short time, while other vessels may be measured periodically to monitor the growth of bacteria. The methods described in this disclosure may be used to detect if bacteria are alive or dead and confirm if, for example, that food processing equipment has been properly sterilized. In some embodiments, the total amount of live and dead organisms will be of interest, and in other embodiments the detection of live bacterial will be the preferred output of the analysis. The disclosed methods may, in some embodiments, selectively detect live bacteria.

As described above, the disclosed methods make use, in some embodiments, of magnetic particles functionalized with recognition elements (e.g., moieties). Non-limiting examples of the recognition elements are antibodies, proteins, oligonucleotides, DNA, RNA carbohydrates, glycoproteins, lectins, or synthetic receptors may be used to bind to biological species of interest. Magnetic particles have been used to capture bacteria by attachment of antibodies to their surfaces. In this way particles may be added to a sample, and then collected using a magnet to capture the particles that have bound to bacterial through an antibody antigen interaction. These particles may be released on a growth medium and after culture the number of colonies may be counted to give a measure of the bacterial. These studies are slow and generally require visual detection of colonies. They do have the advantage of differentiating bacteria that are alive from those that are dead, as only live bacterial will grow in culture. More efficient methods to monitor the growth of bacteria in real-time, with selective detection thereof, may provide considerable benefit to society.

The methods in this disclosure are ideally suited, in some embodiments, to detect biological species (analytes) that have what is known as multivalency. The term, multivalency refers to the fact that there are multiple sites that may be binding sites upon which receptors, ligands, or other recognition species may associate with the biological analytes. If there is only one binding/interaction site that interacts with a specific receptor/recognition element, then the interaction is said to be monovalent. If there are two or more binding sites for a specific receptor then the interaction is said to be multivalent. The interacting sites may be the same or different. Hence, although there may only be a monovalent interaction with one receptor, a multivalent interaction may occur if there are one or more other receptors capable of interacting with different sites on the biological species. Cells, bacteria, and viruses are generally capable of multivalent interactions. Many proteins are also capable of multivalent interactions, either by virtue of their large size that presents multiple independent epitopes (binding sites), or because they are associated in a multimer for binding of recognition elements. An example of a relevant multimers are Shiga toxins, which are highly toxic proteins that may be secreted by certain Escherichia coli (E. coli) serotypes. The B subunits of these toxins of are pentamers and hence provide mutivalency. Additionally, it is possible that multiple recognition elements may be added and that one could also bind the A subunits of a Shiga toxin and expand the mutivalency. There are advantages to having multiple recognition elements in a detection system as it may provide for more robust recognition and differentiation between closely related species. For example, the disease COVID-19 is caused by a specific coronavirus, but there are other known coronaviruses. Methods that selectively detect the coronavirus responsible for COVID-19 will be accomplished by using recognition elements that selectively recognize epitopes associated with the target virus analyte. Using multiple recognition elements could be used to detect proteins that form heterodimers, by recognizing epitopes on each of the proteins involved in the complex. It is also possible that a single protein may be simultaneously bound to a ligand for which the protein is a receptor and a receptor that recognized and epitope on the protein.

Viruses, such as a coronavirus have a protein shells that provide structure and function necessary to invade host cells. These protein shells have many copies of given proteins that assemble into structures, including the capsid that surrounds and protects the viral genome. The multiple capsid proteins and viral coat proteins all provide potential recognition sites for binding. In the case of a virus, there could be a specific epitope, on each of the capsid proteins. There could in also be multiple recognition sites, on each capsid protein. Similarly, the viral coat proteins may present multiple epitopes. As a result, if a recognition group such as an antibody or engineered protein receptor that is specific to one of the epitopes is added, it is possible for it to bind to multiple sites.

It is also possible that virus particles may be detected by their RNA. There are variety of ways that assemblies of RNA or DNA may be assembled and interact with complementary recognition elements. Those who are skilled in the art will recognize that RNA or DNA may be recognized by binding of a complementary oligonucleotide, DNA or RNA. It is possible in some embodiments, to bind to multiple segments a specific RNA, DNA, or oligonucleotide by designing more than one complementary oligonucleotide, DNA or RNA binding unit.

In some embodiments, the biological detection methods described in this disclosure are enabled by the use of particles that contain magnetic, luminescent, or reflective properties or combinations thereof. The particles are functionalized with recognition elements (receptors, ligands, nucleotides, or the like) that bind a biological species of interest. Combinations of magnetic and non-magnetic particles that may be assembled by multivalent interactions with biological analytes provides the basis of a detection event. In some embodiments, the colocalization of combinations of magnetic and non-magnetic particles with a magnetic field provides for a positive detection event. In some embodiments, the localization of only the magnetic particles provides for a negative detection event. In some embodiments, only magnetic particles need be localized, but in this case an additional optical signature associated with the biological analyte is needed. In some embodiments, this optical signature may be intrinsic to the analyte and in others an optical signature may be added. A non-limiting example of the later would be to add fluorescent recognition element, such as a fluorescently labeled antibody that binds to the analyte. There are a diversity of materials and recognition elements that may be used with this method.

The magnetic particles may have inorganic cores that are functionalized on their surface with a coating. This coating may be attached to the inorganic material by a variety of methods. For example, it may be an organic protein may be attached through a phosphate, carboxylate, amine, silicate, polymer, and the like. The magnetic cores of the particles may be small enough that they may become magnetized (magnetic dipoles aligned) the presence of an applied magnetic field and then relax to a random organization in the absence of a field. Materials displaying this behavior are known as superparamagnetic particles. The size of the magnetic domains and the strength of interactions between particles is integral to observing this behavior. In some embodiments the magnetic particle cores are composed of Fe₃O₄ which is known as magnetite. In some embodiments the magnetite cores of the magnetite cores are between 3-30 nanometers (nm) in diameter. Larger particles may retain magnetism and such properties may lead to unwanted aggregation of the particles in the absence of a magnetic field. One who is skilled in the art will realize that there are a number of types of magnetic materials and that nanoparticles may have other compositions. The size of the particle used to exhibit superparamagnetic properties may also change with the composition. The total size of the magnetic particles may be much larger than the cores responsible for the superparamagnetic behavior. For some embodiments, the magnetic cores may be placed in polymers to give particles that may 100 nm to 100 micrometers in diameter.

An exemplary advantage of the superparamagnetic particles is that they do not have strong attractions to each other in the absence of a magnetic field. Larger particles that have persistent magnetization display ferromagnetic behavior, and may have interparticle interactions that cause them to associate. In this disclosure we are interested in particles that have superparamagetic interactions or only transient persistent ferromagnetic interactions that relax over time after the removal of an applied magnetic field. In some embodiments the magnetic particles will have small molecules bound to their surfaces that provide stability, dispersibility in water, reactive groups for functionalization. In some embodiments, magnetite particles may be functionalized by 2-aminoethyl phosphonic acid, which produces a stabilized particle coated with primary amines. It is possible to assemble a polymeric material around an individual or cluster of magnetic particles. This may be accomplished by condensation of siloxanes on the magnetic particles through a condensation reaction, by crosslinking a polymer shell assembled at the surface of magnetic particles, absorption of a polymer from solution onto the surface of a magnetic nanoparticle, by polymerization of a monomer dispersion containing one or more magnetic particles, by assembly of other particles onto the surface of a magnetic particles, and/or by absorption of proteins on the magnetic nanoparticles. These methods may be used to coat a single magnetic particle or create a larger particle containing more than one magnetic particle. It is also possible that more than one type of magnetic particle may be included in a given composition. In some embodiments, materials may be used to produce more complex assemblies that may be controlled as a result of the magnetic dipole dynamics and the magnitude of the magnetization.

In some embodiments, it is of interest to add other functionality to magnetic particles. For example, attachment of an emissive dye to the surface of a magnetic particle may be useful for different optical detection methods. Emissive materials may be incorporated within polymers, either covalently of non-covalently, that surround magnetic particles. Emissive materials may also be directly linked to functional groups, including amines, carboxylates, reactive esters, alkynes, tetrazines, and trans-cyclooctenes attached to the surface of the magnetic particles. In some embodiments, different emissive dyes with different emission colors (wavelengths) may be used to identify particles having different recognition elements. In this way, a multiplexed biological assay may be created that provides for the ability to obtain more information about the biological analytes. It is also possible to use particles capable of reflecting light. In this case interference effects within the particles may selectively reflect light of given wavelengths. Particles having multiple components with different refractive indexes and interfaces may be used to produce reflections. These include assembled block copolymers, chiral nematic liquid crystals, and multiphase colloid particles. To create highly robust methods there will be advantages to having these materials in a solid or polymer stabilized form. For example, a chiral nematic liquid crystal is technically a liquid, however the helical photonic structure of these materials may be preserved after polymerization of some or most of the molecules constituting the liquid crystal. In so doing a reflective solid or semi-solid (gel) particle may be produced.

The non-magnetic particles may range from functionalized polystyrene particles that 10 nm to 300 microns in diameter. In some embodiments, particle like constructions may be used wherein a receptor is covalently modified with one or more molecular of polymeric emissive dyes. The latter may not be conventionally considered as a particle, but may provide the same function of a particle and one who is skilled in the art will recognize that this approach may provide the same emissive labeling to the analyte as a particle. In some embodiments polymeric dyes may provide for efficient optical absorption and emission properties. In some embodiments these polymeric dyes may be a water-soluble conjugated polymer. Non-limiting examples of water-soluble dyes are certain polyfluorenes, polyfluorene copolymers, and polyfluorenes that have other dyes attached to them. Conjugated polymers may be used as efficient antennas for the absorption of light and transfer the energy to minority sites in the polymer or pendant dyes to create longer wavelength emission, than would have been expected from the polyfluorene backbone. In some embodiments, the non-magnetic particles will be designed to scatter or reflect light.

In some embodiments, non-magnetic particles may be produced by the assembly of non-water-soluble materials by different dispersion methods. For example, adding a tetrahydrofuran solution of dye-polymer mixture to water that is being vigorously stirred, vortexed, and/or being stimulated ultrasonically may produce a dispersion. For some embodiments, the materials undergoing dispersion will benefit from the addition of surfactants that stabilize the dispersions in water. The surfactants may be small molecules, particles, biomolecules, or polymers. The particles may be solids composed materials that are in a crystalline, glassy, amorphous, rubbery, plastic, or combinations thereof. In some embodiments, these phases may be interspersed with fluid phases internal to the particles. In other embodiments, the particles are fluids. A fluid particle may allow for dynamic assemblies of the surfactants and other functionality associated with their surfaces. In some embodiments, the ability to cluster recognition elements around the binding of an analyte will be desirable. Clustering of recognition elements may be used to enhance the strength of the association of the particle with the analyte and may also be used to create a specific optical signature. In some embodiments, clustering may enhance energy transfer processes that may provide a new enhanced optical signature associated with analyte binding. Particles may have clear domains and structure. In some embodiments, particles will have what is known as a Janus structure wherein there are in effect two separate domains that define two different faces to the particles. The different phases may be solids or liquids and may have different optical elements. In some embodiments, each phase will have a different luminescent dye. In some embodiments, at least one of the phases will have a dye that absorbs light and is largely non-emissive.

In some embodiments, microfluidic methods may also be used to make precision particles containing dyes or have multiple intraparticle phases encoding different optical information. In some embodiments these particles will range in size from 1 micron to 300 microns. In some embodiments, there may be two phases within the particle that have emissive optical elements. In some embodiments one of these phases may be a polymer and one phase may be a liquid. In other embodiments, both phases may be polymers, and in yet other embodiments both phases may be liquids. Block copolymers capable of assembling into structures capable of reflecting light may similarly be dispersed to produce particles. In some embodiments, the polymers may be the dyes and in preferred structures the polymer is a conjugated polymer. A base polymer may be chosen to very efficiently absorb light and produce luminescence. In some embodiments, a secondary and minority chromophore may be incorporated either covalently or non-covalently into the polymer. If the minority chromophore is emissive and capable of accepting energy from the base polymer, then the emission wavelength may be shifted. A general requirement for such a shift in wavelength is that the emission associated with the addition of the chromophore may, in some cases, be longer wavelength (lower energy) than the emission of the chromophore donating the energy. The degree to which the emission shifts to lower energy or longer wavelength may vary. The mechanisms upon which energy may transfer between chromophores may involve dipolar coupled processes, known as Förster energy transfer or mechanisms that provide for strong electronic coupling including what is often referred to as Dexter energy transfer.

In some embodiments, the core of a luminescent particles may be substantially, or completely, composed of molecular or polymeric chromophores. For example, the 7C-conjugated polymers below may be dispersed in water with surfactants to create stable luminescent particles. An advantage of these materials is that they may have broad absorbances allowing for efficient excitation by a variety of light sources. The “R” groups in these structures may be independently varied and are known to one skilled in the art as indicating that a wide variety of organic groups may be attached to the structures, included by not limited to H, alkyl groups or aromatic groups. Alternatively, R may be a halogen in the case that it is bound to a carbon atom. In some embodiments, some of the R groups will be hydrophobic and some will be hydrophilic. In some embodiments, the polymers may behave as their own surfactants. In some embodiments, some of the R groups may contain reactive elements including, but not limited to, amines, carboxylates, tetrazines, trans-cyclooctenes, cyclic alkynes, maleimides, thiols, disulfides, reactive esters, or norborenes. In other embodiments the conjugated polymers are part of a macromolecular structure known as a block or brush polymer. In a block polymer, a polymer has multiple sequences and the conjugated polymers may be flanked on one or both ends by a block polymer that may provide surfactant characteristics and/or reactive elements. In some embodiments block copolymers structures may be used to stabilize the aqueous dispersions of the conjugated polymers. In some embodiments the non-conjugated block of the block copolymer structure has additional dyes. In some embodiments the non-conjugated block of the block copolymer structure has hydrophilic character. In some embodiments the non-conjugated block of the block copolymer structure has reactive groups including, but not limited to, amines, carboxylates, tetrazines, trans-cyclooctenes, cyclic alkynes, maleimides, thiols, disulfides, reactive esters, or norborenes.

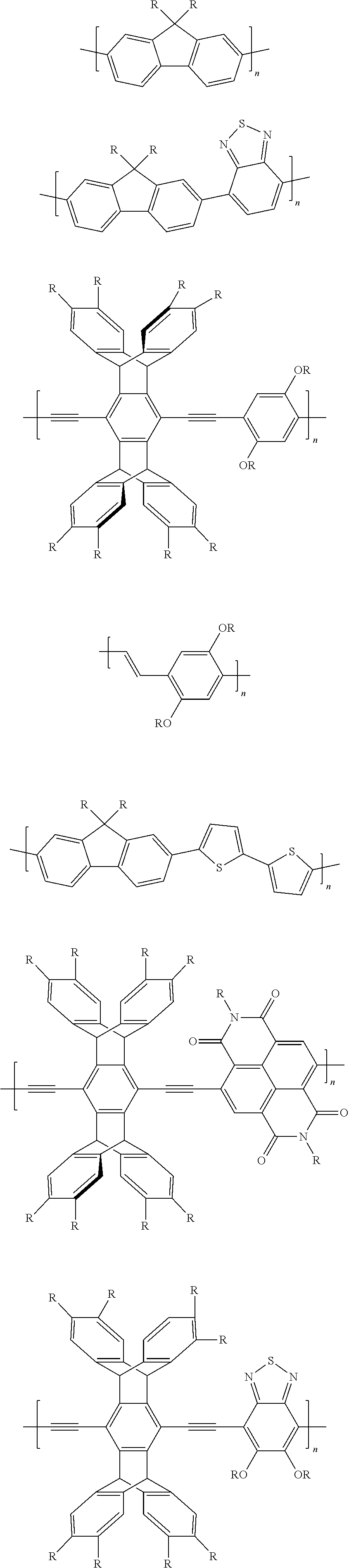

Conjugated π-conjugated polymers are generally efficient at energy transfer (migration) and the locally generated excited states may migrate throughout a particle. In this way the excitations will tend to transfer to lower energy states that may be introduced by either covalent of physically mixing in a structure that has a lower band gap. In some embodiments, this method may produce materials that have a large separation from the excitation and the emission that may be an advantage to measurements wherein excitation light needs to be excluded or filtered from the detection. Energy migration may occur in mixtures of materials. This may involve molecular and polymeric chromophores. The materials shown that have the large 3-dimensional pentiptycene unit in the backbone are particularly well suited to form stable compositions with other polymers or molecules as a result of the free volume promoted by structure. The materials that are shown below all have a majority portion of the composition that has a higher band gap and a minority portion of the composition that has a lower band gap. In the structures shown y>x and R has the usual meaning to one skilled in the art. An advantage of this embodiment is that the majority portion of the composition may be uniformly excited and the wavelength where the majority emission occurs is dominated by the minority portion of the composition.

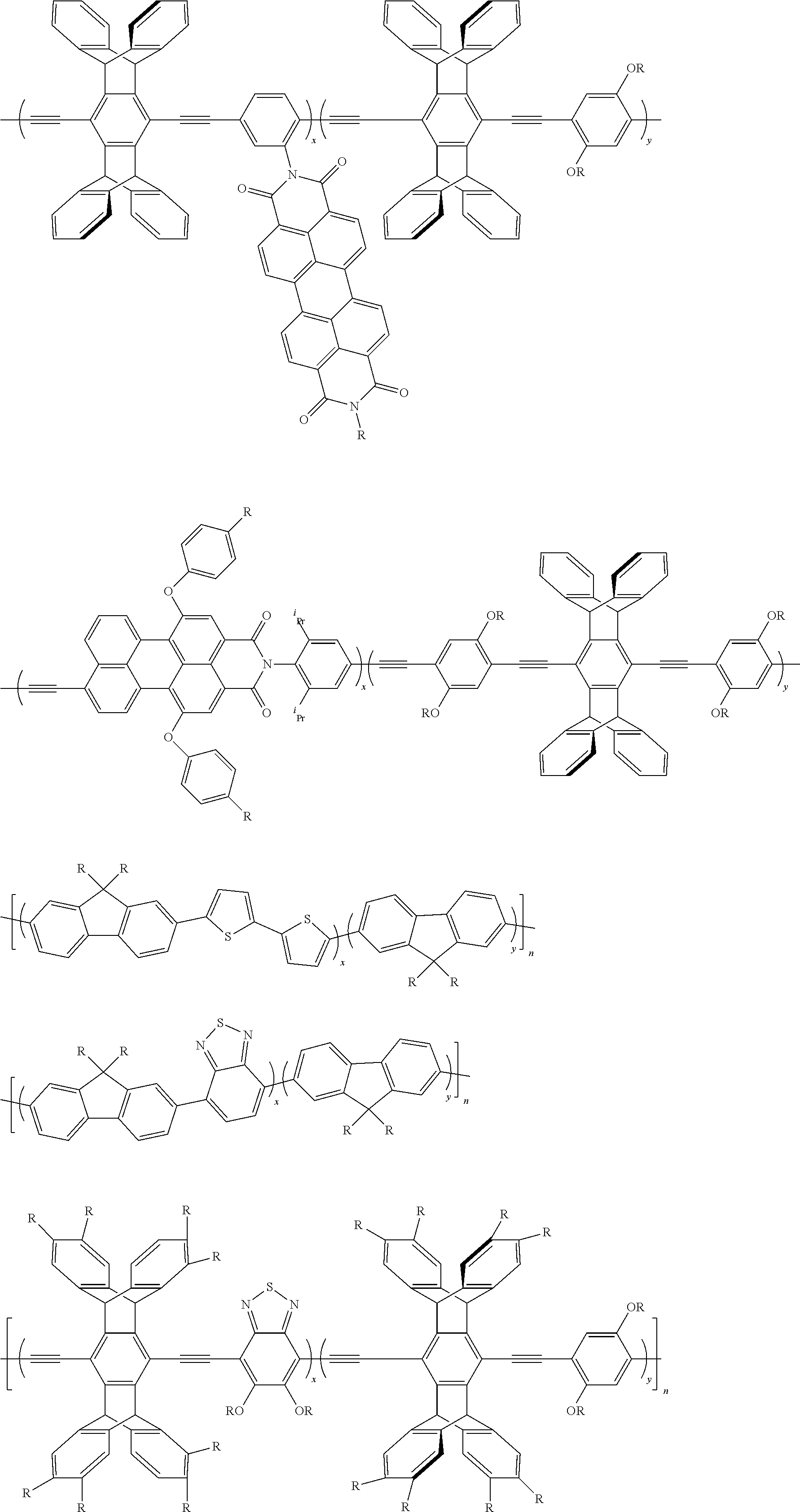

In some embodiments, it will be advantageous to make use of luminescent optical elements in the particles that undergo delayed emission. This process allows for a light to be detected after a delay from the excitation. The advantage is that other fluorescent signals will not be present after the delay. It is possible that fluorescent and delayed emission signals may be used together to provide additional information. Materials that display delayed emission include metal phosphors and organic molecules displaying thermally activated delayed fluorescence. Well known metal phosphors of particular interest are those based on gold, iridium, and platinum organometallic compounds and coordination compounds based on ruthenium, europium, and terbium. Another type of material that may undergo delayed emission are materials that have singlet and triplet states that are in thermal equilibrium. These materials undergo what is called thermally activated delayed emission (TADF) and the luminescence from these materials is delayed because of the fact that upon excitation the singlet state undergoes intersystem crossing to the triplet state which has a very long lifetime and hence very slow radiative rate. The triplet state and singlet state thermally equilibrate and the higher radiative rate of the singlet state will result in emission. However, the fact that the majority of the time is spent in the triplet state, the emission lifetime is longer than would be expected by the radiative rate of the singlet.

The cores of luminescent particles, in some embodiments, will be hydrophobic and to create stable suspensions in water and afford efficient bioconjugation, surfactants and/or reactive elements are useful. In some embodiments, these surfactants are polymeric in nature and may contain reactive elements including amines, carboxylates, tetrazines, trans-cyclooctenes, cyclic alkynes, maleimides, thiols, disulfides, reactive esters, or norborenes. Non-limiting examples of polymeric surfactants are shown below that may provide for bioconjugation directly of be activated to allow for bioconjugation. In some embodiments, it will be useful to use chemistry to link the polymeric surfactants with multifunctional groups or polymers. These groups may in come embodiments provide additional sites for bioconjugation or for adding groups to ensure that the particles do not display non-specific bonding to biological species, particles, or surfaces. It is also possible to use biomolecular recognition elements to functionalize particles. In some embodiments, a biotin may be attached to a particle and a functionalized streptavidin may then bind to the surface. Alternatively, streptavidin may be bound to the surface and a functionalized biotin may bind to the surface. In other embodiments, Protein A may be bound to the surface of the particle which will bind to an antibody.

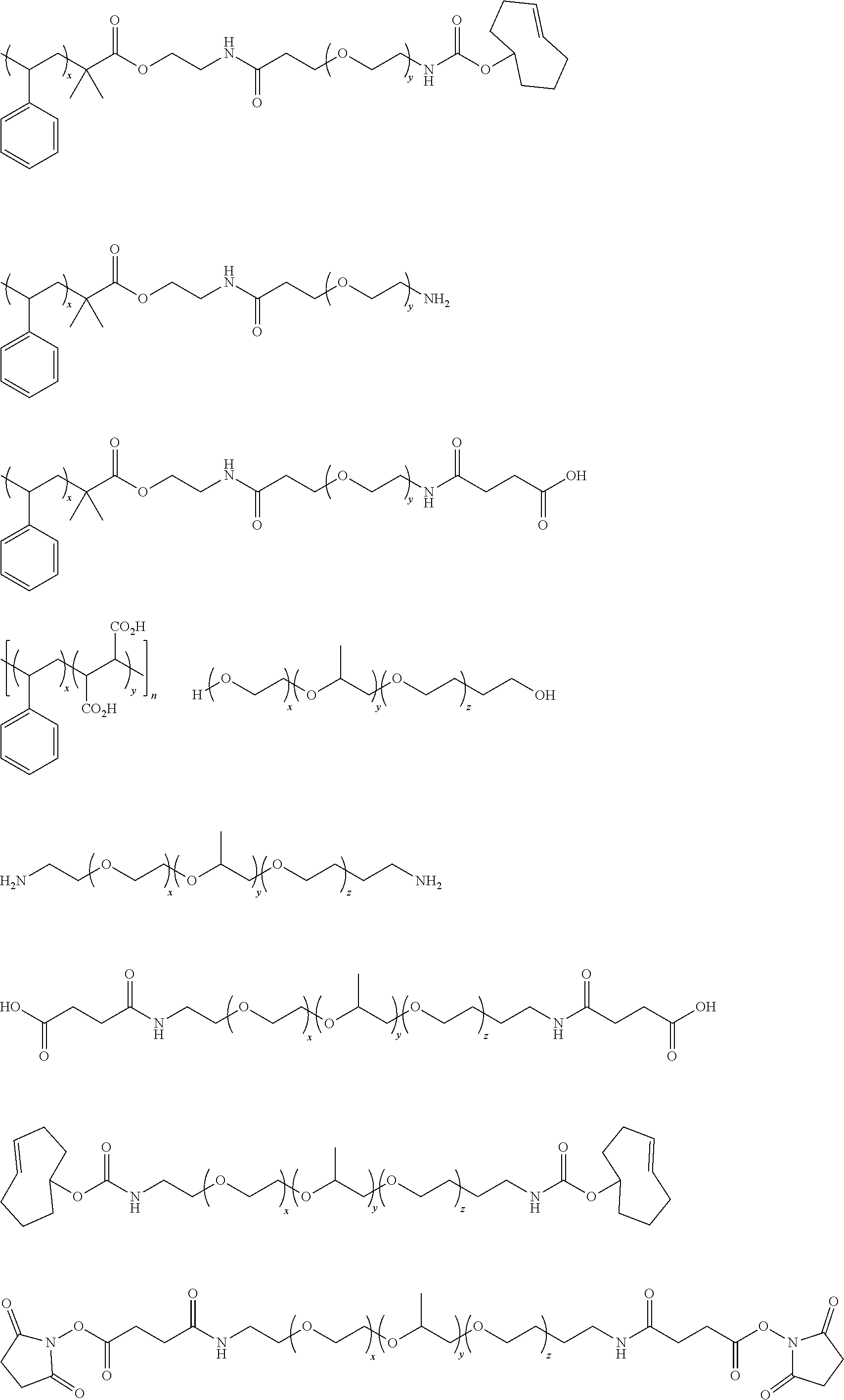

In some embodiments, reflection or scattering from particles will be used to indicate the presence of the analyte. The wavelength of light reflected or scattered by a particle may depend on the nature of the particle. It may depend on the composition and if metal particles, oxides, the angle of interfaces, or periodic structures. In the case of particles created from chiral nematic liquid crystals the helical pitch determines the wavelength of reflected light. The type and amount of the chiral molecules causing the twisting of the liquid crystal molecules determines the pitch and the wavelength reflected. In some embodiments, photonic block copolymers will serve as reflectors, the structures, spacing and refractive index contrast between the blocks may change the wavelengths reflected. In other embodiments, particles may reflect light as a result of curved interfaces between two different refractive index materials. Curved interfaces may be used to reflect light in this regard. In the case of reflective materials, the angle at which the active photonic elements are oriented with regard to the exciting light and the direction at which the light is detected may give rise to changes in the efficiency of the reflectivity and the wavelength. In other embodiments metal nanoparticles, such as gold nanoparticles, are used to scatter light and create an optical signal.

In some embodiments a multiplexed assay using a mixture of different luminescent materials or reflector encoded particles with associated different receptors, may be used to detect multiple analytes at one time. Such an assay may be used to differentiate between concentrations of closely related species, or detect simultaneously multiple species. For example, in bacterial detection it could be possible to create an assay that detects within a single vessel all relevant pathogenic organisms of interest for a particular food production process. Similarly, it could be possible to have a test that simultaneously detects for influenza viruses that cause Flu A and Flu B, but also detect the coronavirus responsible for the disease COVID-19. Mutiplexed assays may contain multiple encoded magnetic particles and/or multiple encoded non-magnetic particles with signaling entities. The recognition elements (e.g., moieties) on encoded to specific particles may similarly be diverse and non-limiting examples include antibodies, proteins, oligonucleotides, DNA, RNA, carbohydrates, glycoproteins, lectins, virus capsids, or synthetic receptors.

In some embodiments, the density of the materials that make up the non-magnetic particles with signaling entities may be important. The non-magnetic particles may be either more or less dense than the water phase. In some embodiments, non-magnetic particles with signaling entities that are not associated with a magnetic particle will not be captured at a location determined by an applied magnetic field and will either sink to the bottom of a vessel of float to the top of the solution. The differences in density will also be a factor and may be used to ensure that particles do not localize at the site of the magnetic field if they not bound to a recognition element. In some embodiments some gentle mixing may also help to remove particles. In some embodiments, the rate at which the particles float to the surface or drop to the bottom of a vessel is important and may affect the rate at which each measurement cycle may be performed. It is generally advantageous for measurements to be made as fast as possible. Methods to create more buoyant particles include creating porosity within the particles by generating air pockets or free volume. In some embodiments, extraction of a non-polymerized material from a polymer particle may provide pores capable of creating air pockets and/or free volume. In this case it will be important that the pores have hydrophobic character so that they do not fill with water. Incorporation of molecules that have elements such as halogens or metals may be used to create particles that have higher densities. One skilled in the art, will understand materials that pack densely or have heavier elements may produce materials with higher densities.

In some embodiments, measurements will be performed on the sidewalls of a vessel. In this case the emission will be monitored on the sidewall of the vessel. To minimize the background emission in a measurement from a point on the sidewall of a vessel, the emissive non-magnetic droplets may either float to the top or sink to the bottom. This process is shown in FIG. 1B.

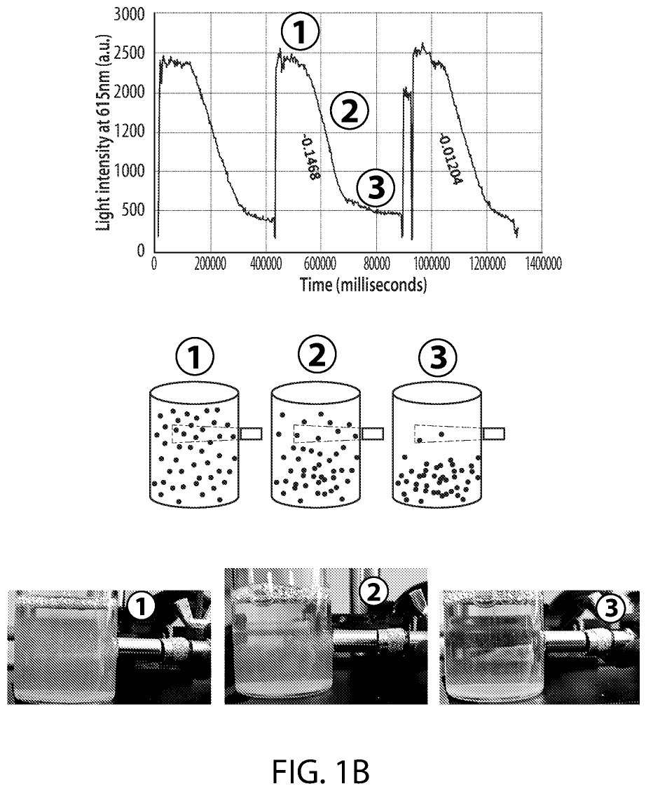

Larger particles generally tend to sink faster than smaller particles. This effect is clearly illustrated in FIG. 2. In this embodiment, droplets are composed of diethyl phthalate which has a density of 1.12 g/cm 3. In some embodiments particles of a larger size will be used to cause the non-magnetic particles to sink faster. For different applications there may be an optimal combination of density and size of the non-magnetic particles containing signaling entities.

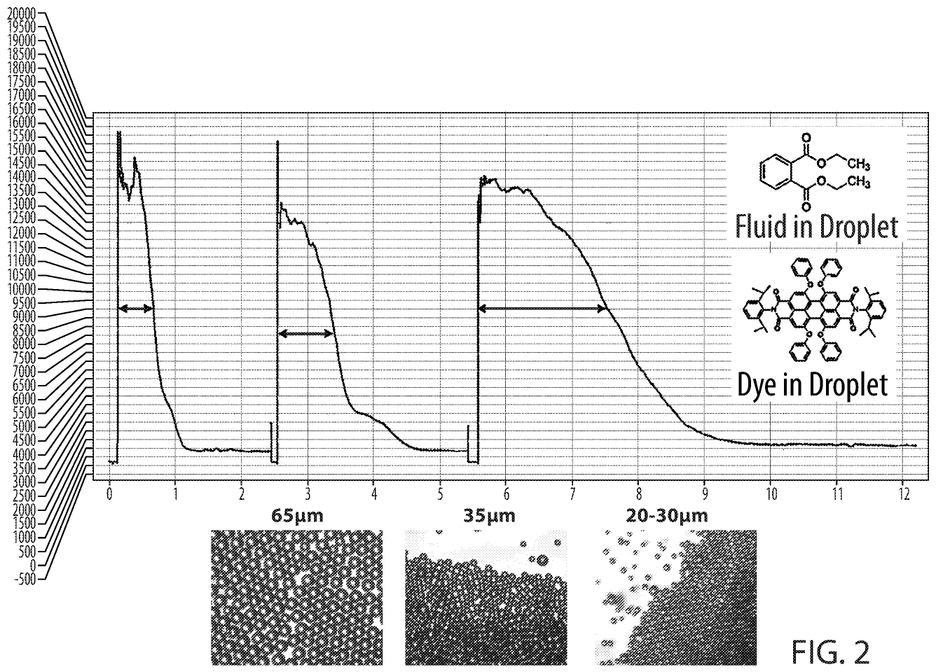

To detect a biological species, particles will may to be linked (conjugated) to a recognition element (e.g., moiety). The recognition element may be considered as either a receptor or a ligand. A receptor typically binds around (partially or completely engulfs) its complementary ligand. In biological recognition, the site that the antibody binds to is generally referred to as epitope. In the more general description of biological recognition and epitope is generally referred to as a ligand, and an antibody or equivalent binding element is called a receptor. In some embodiments, ligands may be used to detect receptors and/or a species associated with a receptor. That is the receptor may be the analyte. Alternatively, receptors may be used to detect ligands and/or species having ligands. In this case the ligand or a species associated with the ligand are the analyte. For example, an antibody may be used to detect a species containing the target antigen. However, the antigen or a species having the antigen may be used to create a method to detect specific antibodies. Receptor-ligand interactions are often referred to as having lock and key interactions. This physical analogy reflects that the lock surrounds the key and that there is a special recognition between the lock and the key. In some cases, it may be or interest to have magnetic or non-magnetic particles containing signaling entities without recognition elements present as a control element. In some embodiments, the presence of these species may indicate that the solution has been prepared properly and non-specific association of these groups to magnetic or the non-magnetic particles, may indicate that there is a problem with the assay or a complexity with the sample. For an assay to recognize a particular analyte, it may be useful that at least one magnetic particle and one non-magnetic particle contain a recognition element that will associate with the analyte. The recognition element may be the same on the magnetic and non-magnetic particle, provided that the analyte is multivalent may bind more than one recognition element. If these particles are placed in solution with the analyte, an association may take place. A non-limiting example of an analyte mediated assembly of magnetic and non-magnetic particles is shown in FIG. 3. Although not shown in FIG. 3, the magnetic particle may have an emissive (luminescent, scattering, or reflective) element that may be distinct from that of the non-magnetic particle.

It is possible to have magnetic and non-magnetic particles with different recognition elements assemble around a mixture. The interactions may be polyvalent or monovalent in nature and may involve ligands as well as receptors.

In some embodiments, antibodies will be used as the receptors to bind to antigens associated with the analytes. Antibodies may be mono-clonal or poly-clonal. A monoclonal antibody will bind to a specific epitope, which may be a substructure of the antigen. In many cases the antigen is a protein that may be monomeric or multimeric, it may be excreted or bound to the surface of a cell, organism or surface. A polyclonal antibody is a mixture of antibodies that broadly recognize a target. These receptors may bind to multiple epitopes and protein antigens. Antibodies are large biomolecules with molecular weights typically of the range of 150,000 Daltons. In some embodiments, it is useful to have receptors that have lower molecular weight. One type of receptor is derived from what are known as nanobodies or as single-domain antibodies that are derived from natural camelid antibodies. The nanobody structure referred to also as VHH are only a fraction of the size of an entire immunoglobulin (IgG) antibody. The smaller size of these receptors may allow for improved stability, less tendency to aggregate, and lower production costs as compared to antibodies. Other families of proteins may be used to create receptors and one class of thermal stable protein receptors are the reduced-charge Sso7d (rcSso7d) engineered proteins. Protein receptors may be identified by using selection processes from large libraries and in these methods the receptors are selected for their binding to specific antigens or structures associated with the target analytes. In some embodiments, the genes encoding the final protein receptors may be used in bioproduction of these materials. In these processes structures may be added that may allow for facile attachment (conjugation) to particles. Lectins are another class of receptors that may provide recognition and these receptors bind to carbohydrate containing molecules. Carbohydrate molecules may have complex structure and cells or organisms may be recognized by the complex carbohydrates that are attached to their surfaces. Protein nucleic acids may also be developed to bind molecules of interest. Additionally, there are many small molecule receptors that may be used to bind to analytes of interest.

In some embodiments, assay may be produced if the particles are functionalized with ligands that will bind to receptors that are associated with the analyte. In some embodiments, the ligands may be carbohydrates and the analyte may be bacteria. In other embodiments, the ligands may be carbohydrates the analyte may be a virus. In some embodiments, influenza virus may be the analyte and molecule containing a sialic acid group is used as the ligand.

In some embodiments, nuclei acids are the analytes and are also the recognition elements. A system designed to detect a specific DNA or oligonucleotide sequence may be produced provided that the magnetic and luminescent particles are both functionalized with sequences that may bind to the target DNA/oligonucleotide, or a hybrid complex thereof, at the same time. In some embodiments binding the magnetic particle to one nucleotide sequence and the luminescent particles to a second nucleotide sequence may provide for increased specificity because two different features may, in some cases, be recognized at the same time.

In some embodiments, individual particles may have multiple recognition elements. The recognition elements may be chosen to recognize a specific analyte and multiple recognition elements may target different features of an analyte. In some embodiments, multiple recognition elements may be used to impart a broader scope of the analytes that the particles may be used to detect. In some embodiments, particles may be designed to recognize classes of bacterial or potentially all bacteria. In other embodiments, particles may be designed to recognize related classes of viruses.

In some embodiments, the functionalization of particles with recognition elements may be performed as part of the initial particle assembly. For example, the recognition element may be associated with a polymer or surfactant that assembles on the surface of the particle during the dispersion process. In some cases, the recognition element may provide surfactant characteristics that stabilize the particle during its formation. In some embodiments, it is desirable to be able to produce particles having different magnetic and optical properties that may functionalized at a later stage with recognition elements. In so doing it is possible to be more efficient in creating different particles for specific applications. In some embodiments, one type of base particle may be functionalized at a late stage. In some embodiments, particles may be functionalized immediately before using in an assay. In other embodiments, particles may be functionalized in situ in the assay. Late stage functionalization may allow for more sensitive recognition elements to be added just prior to an assay.

There are many suitable methods for covalently linking recognition elements to the surfaces of particles. These include the formation of amide linkages, thiol additions to alkenes, nucleophilic aromatic substitution, cycloaddition of organic azides to alkynes, Diels Alder reactions, photochemical reactions, condensation of aldehydes with amines, enzyme catalyzed reactions, and the like. In some cases, non-covalent methods may be used. Biotinylated particles may be bound to receptors via noncovalent assembly with streptavidin. Oligonucleotides may be used as recognition elements as well as a method to bind other elements to particles. The surface chemistries of the particles may be chosen by design to allow for the efficient attachment of receptor groups. Additionally, attachment of Protein-A to a particle may be used as a general method to non-covalently attach specific types of antibodies.

In some embodiments, the density of receptors on the surfaces of the particles may be adjusted for optimal performance. In general, it is important to have a sufficient number of receptors on the surface of a particle to efficiently bind a target biological species. The interactions between the particle and the target analyte may be multivalent. A monovalent or weak interaction between a given particle and analyte, may result in binding to multiple receptors on the surface of a particle may increase the probability of a binding interaction upon an encounter of the particle and the analyte. In so doing a first point of attachment will direct a stronger binding. The presentation of the receptor on the particles surface is also often important. In many cases it is important to have a water-soluble linkage between the receptor and the particle. This will allow for the receptor to not be adversely affected by the surface of the particle, which is some embodiments will be hydrophobic. Such a tethered linkage may also allow the receptor freedom to assume geometries useful for optimal interaction with the target analyte. In some cases, the water-soluble linkage between the particle and the receptor is based on poly(ethylene gloycol) or a PEG group that has integer numbers (n) of (CH₂CH₂Oₙ between the receptor and the particle. Other linkages are also possible and a polymer may be anchored to a particle that has a hydrophilic region that may be used to bind multiple receptors. An example of a linkage are systems derived from block copolymers of polystyrene and polyacrylic acid. This polymeric surfactant may be functionalized by conversion of some or all of the carboxylic acid groups to reactive esters that react with amines to produce sites that may be used to link a specific receptor.

In some embodiments, the reactive groups associate with the receptor may be used to react with the surface functionality of the particles. Reactive amines in a receptor may react to form amides with reactive esters. Thiols that are attached to a receptor may be add to reactive alkenes. Carbohydrates attached to a receptor may be bound to boronic acids or oxidized in situ and reacted with amines. In many cases it is advantageous to make use of what are known as click reactions, that have bio-orthogonality. These reactions are highly specific and efficient in complex environments. A particle could be modified with a tetrazine and a receptor may be modified with a trans-cyclo-octene. These two materials may then react through a Diels Alder reaction to produce stable linkages. Alternatively, the particle may be modified with a trans-cyclo-octene and the receptor may be modified with a tetrazine to do the same process. Other click reactions involve organic azides and alkynes. Terminal alkynes may react in click reactions through a copper catalyzed process. However, copper ions are toxic and in some cases a copper free click reaction is useful wherein a cyclic strained alkyne is used and may undergo a cycloaddition reaction with an organic azide without a copper catalyst. The latter are referred to copper-free click reactions.

In some embodiments, antibodies are functionalized with multiple tetrazines that react with trans-cyclo-octene on the surfaces of the magnetic or luminescent nanoparticles. In some embodiments, amines on the antibodies are reacted with active esters to attach the reactive functionality. The reagents may also be reversed wherein the trans-cyclo-octene is added to the antibody and the tetrazine is attached to the particles.

The precision of the attachments between the receptors and the particles may also be important. In some cases, multiple click reactive groups may be added to a receptor without compromising its binding properties. In other cases, it may be important to have a specific connection to the particle that is remote the active binding site of the receptor to produce the most effective binding to the target analyte. In some materials such as protein receptors that may be produced by recombinant methods, it is possible to encode for specific functional groups such as series of amines or thiols at a particular location of the receptor to allow for attachment to the particle of the attachment of a click reactive group. It is also possible to use highly selective enzyme reactions, for example on the carbohydrate portion of an antibody, to install functional groups.

The methods in this disclosure may make use of a wide array or recognition elements. A particular assay may make use of one or more different recognition elements. There are embodiments, wherein a weakly associating recognition element may be paired with a stronger binding element to produce a cooperative effect.

The methods described in this disclosure are generally useful, in some embodiments, for the rapid detection of biological species (the analyte) by either a luminescent or other optical signal generated at a location defined by a magnetic field. The method involves the use of magnetic particles that become associated with other non-magnetic particle having an optically readable signal through interactions mediated by the biological species of interest. Applied magnetic fields may then localize the magnetic particles along with the biological analyte and other non-magnetic emissive particles at a specific location for optical interrogation. In some embodiments, the location for the measurement in a container is chosen to be a location in which the particles will not naturally localize in the absence of an applied magnetic field. In some embodiments, particles will be made to be either more-dense or less dense than the water based medium that they are suspended. If the luminescent particles are not associated with the magnetic particles, they will either float to the surface of the water-based solution if they have a lower density than water, or sink to the bottom if they have a higher density. In some embodiments, the optical measurements will not be performed at the bottom or top of a fluid within a container. If particles having a readable optical signature (signaling entity) become bound to a biological analyte as a result of a multivalent interaction, then magnetic fields may be used to localize the particles at a location that they would not otherwise be observe to localize as a result of flow of density.

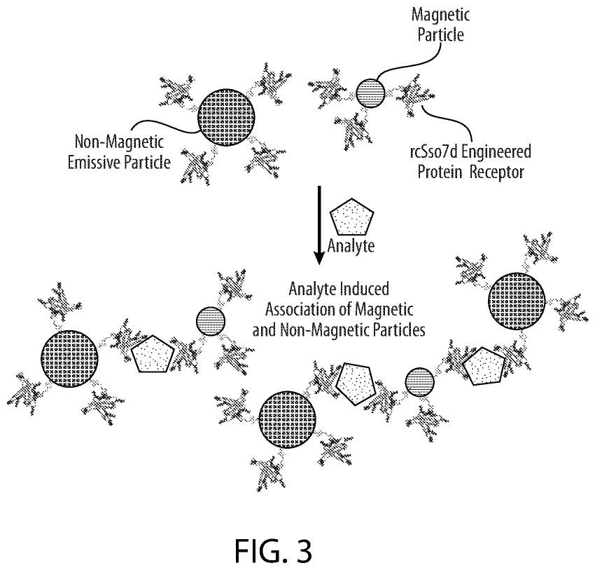

In a measurement to determine analyte, a sample containing magnetic, optionally emissive, particles having one or more recognition elements and non-magnetic emissive particles having the same or other recognition elements that recognize the same analyte or collection of analytes are added to a solution. If a target biological analyte is present multivalent binding to the magnetic particles and the non-magnetic emissive particles will associate as a result of both of them being bound to the analyte. The magnetic particle within this association will cause the collective assembly to be drawn to a prescribed location by application of a magnetic field. The detection of the optical signal from the non-magnetic particles at the location defined by the magnetic field will indicate the presence of the analyte. The optical signal may be from a luminescent dye in the non-magnetic particles or reflection or scattering from the non-magnetic particles. The magnetic particle may also contain an optical signature that will be assembled at the location defined by the magnet in the absence of the analyte. Such a signal may confirm that the proper reagents were added to the assay. In some embodiments, the emissive magnetic particles may provide for a reference signal that may be used to provide a quantitative determination of the analyte concentration. In some embodiments the analysis will be performed at the sidewalls of a vessel containing an analyte as shown in FIG. 4. In some embodiments, the particles are localized by placing a magnet directly against the outside wall of the vessel and the optical measurement is made after retracting the magnet and fiber optic from the wall. In some embodiments this retraction is 1 millimeter to 10 millimeters to allow for improved collection of the optical signal by the fiber optic probe. The magnetic field in this case may be suitably strong to prevent the associated complex of the magnetic particles, non-magnetic particles, and analyte from dissociating from the sidewall of sinking to the bottom of the vessel or floating to the top. In some embodiments, the magnet and fiber optic may not need to be moved and may simply be put proximate to the vessel for localization and excitation to create a detectable signal. In some embodiments, the analyte determination is made as is shown schematically in FIG. 4. The same method may be used to detect analyte with magnetic particles containing a recognition element and an alternate method to add an emissive optical signature indicating the presence of the analyte. In these embodiments a recognition element may be added that has an optical signature. This optical signature may be in the form of an emissive dye bound to the recognition element.

To prevent false positive detections of biological analytes, it will be important, in some cases, to ensure that non-magnetic particles are not physically entrapped with magnetic particles with application of a magnetic field. This latter function may be accomplished by using particles with suitable differences in their density relative to water and their physical size. In general, a sufficient density difference is useful to ensure a sufficient force and rapid flotation or sinking is present to prevent non-magnetic droplets form being assembled at a site defined by a magnetic field. In some embodiments, continued solution mixing (flows) will also serve to dislodge particles that may not be bound by recognition elements and thereby reduce the potential of a false positive signal.

The optical signature of the colocalized particles may be read by a bifurcated fiber optic cable wherein one fiber waveguide contains the excitation and the other fiber waveguide transports light to an optical detector. In some embodiments, particles are initially suspended in a solution by mixing and an applied magnetic field is uses to localize the particles at a location other than that which the particles would naturally assemble based on their density. The magnetic particles will always assemble at the location and may optionally contain a luminescent dye or reflective element to allow for their quantification and to confirm that particles have been collected. The magnetic particles could have been used to extract and concentrated from another solution. If a biological analyte is present that binds to the recognition elements of the magnetic particle as well as the non-magnetic particles bearing an optical signature, then a signal may be obtained at the location wherein a magnetic field localizes the particles as shown below. In this case the bifurcated fiber optical cable is inserted in a magnet with a hole through its center. The signals may of the control and that associated with the detection event may be differentiated based on the wavelength of the luminescence or reflected light, and/or the excited state lifetime. The latter feature may use one or more of the emissive elements having a delayed emission.

In some embodiments, the emission localized magnetic and signaling entities are detected by a detector such as an imaging system or a camera. This detection mechanism may, in some cases, reveal features that may be used to differentiate between non-specific binding events. For example, in some embodiments, a magnetic particle will have one emission and the co-localized signaling entity will have a second and distinct emission. Colocalization may be confirmed, for example, by analysis of an image that can discern the different optical characteristics of the two elements. In some embodiments, the size and shape of an optical signature can be used to reveal information. By way of example and without wishing to be bound by such, if a very low signal is detected but is localized to an object that is much larger than expected for the analyte, it may be that the signaling entities are non-specifically bound to non-analyte containing particles. These non-analyte particles may include, for example, fibers, dirt, plant materials, tissue and the like.

In some embodiments the analyte solution may have emissive signals that may interfere with the emissive single collected from the localized complex of the analyte, magnetic particles, and non-magnetic particles. In some embodiments the solution may be simply removed to reduce this background. The vessel may also be adjusted to place the solution in a different location. This may be performed by tipping, inverting or deforming the vessel. On some embodiments, the localized complex of the analyte, magnetic particles, and non-magnetic particles may be at the bottom of the vessel and held in place with a magnet. Inversion of the vessel, with potentially mild mixing, will cause all of the materials not associated with the magnetic particles though the analyte to flow away from the location of the magnet. The optical measurement may then be made on the material that remains as a result of the magnetic field. In some embodiments, the magnet may be retracted to provide for optimal collection of optical signal by the fiber optic probe, photodiode, or imaging detector.

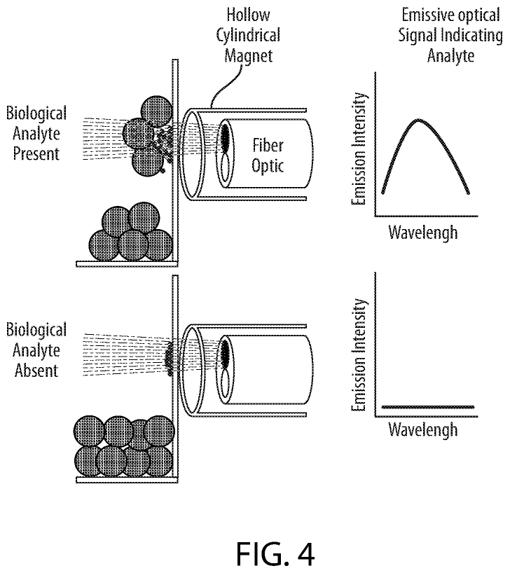

It is also possible to collect the localized complex of the analyte, magnetic particles, and non-magnetic particles in a flow channel. In some embodiments the analyte solution will be flowed between two reservoirs connected by a constrained flow channel. In this configuration the magnet may be on one side of the flow channel and the fiber optic probe may be on the opposite side. The solution may be flow between the two reservoirs on either side of the flow channel once or multiple times to ensure complete localization of the complex of the analyte, magnetic particles, and non-magnetic particles. The light collection by the fiber optic may be enhanced by placing a lens in the optical path as shown schematically for the flow channel scheme in FIG. 5. In this flow scheme, if all of the solution may fit in either of the two reservoirs, then there will be no residual solution in the channel when a measurement is performed, thereby eliminating any potential background from the solution.

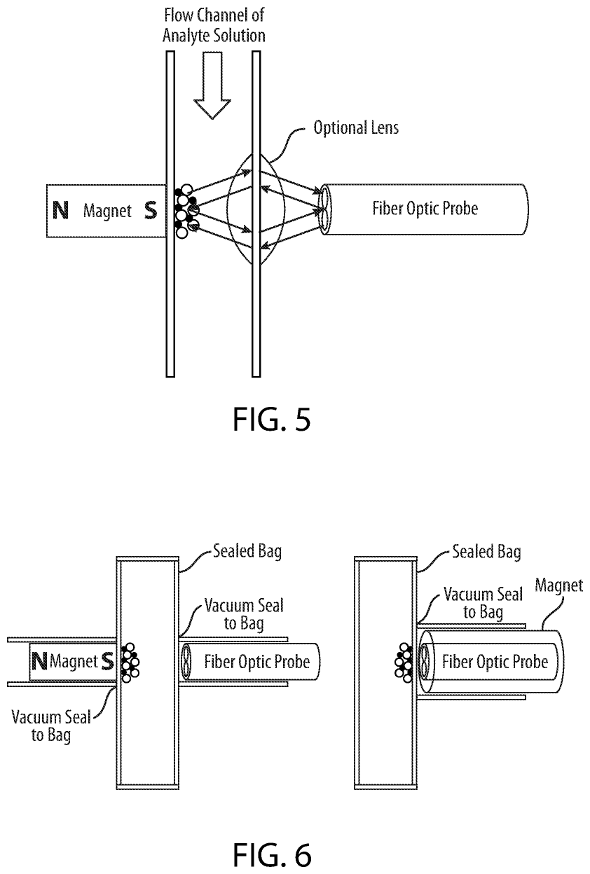

In some embodiments, it the measurements will be made in a bag that may contain the sampling materials. In some embodiments, a sponge that was used to swipe a surface to collect an analyte is placed in the bag. The bag may be mechanically stimulated to affect the release of the analyte from the sponge. In some embodiments, the bag once sealed with the sponge is never opened prior to a measurement to determine if analyte is present. This may reduce potential exposure of the operator to a pathogenic organism and prevent contamination of a work site. The disclosed method may be used to perform measurements in plastic bags provided that the magnetic, non-magnetic particles designed to bind to the analyte are present in the solution. Alternatively, measurements may be made using magnetic particles and an analyte specific recognition element having an optical signature. In some embodiments, the non-magnetic particles are luminescent. In some embodiments both the magnetic particles and the non-magnetic particles are luminescent. In some embodiments the bag may be manipulated to ensure that the measurement is made without background emission from the solution. To make a contact to the plastic bag a suction seal will be made. The magnet may be across the bag from the fiber optic or colocalized with the fiber optic as shown in FIG. 6.

In some embodiments the physical manipulation of the sample vessels will be part of the analysis method. These manipulations may be done manually or with automation. In some embodiments, a mechanical arm, which may be considered as a robot, will be programed to manipulate samples for measurements. In the case of a flow channel, this may involve the act of tipping of inverting a sample. In embodiments wherein the vessels are inverted, this may also be performed by a robot. Similarly the measurements on plastic bags may also be automated with the use of robotics.

Emission measurements generally use an optical excitation and a detector. In some embodiments, the optical excitation may be performed at multiple wavelengths and the detection may be detected over a range of wavelengths with resolution of the signal. Such a device may be constructed from a commercial emission spectrometer. Simpler devices may be also produced wherein the signals are generated by a light emitting diode and the emission is detected by a photodiode. The signals may be collected in a mode wherein the emitted or reflected light is detected while exciting the sample or using delayed emission wherein the emitted light is collected after the exciting light is removed.

The localization by the magnetic field may be key to this process, in some embodiments. Samples are stirred, shook, vortexed, or mechanically mixed in a way that all of the particles are suspended. Once the mixing is ceased the particles will either float to the surface or sink to the bottom. This process may be monitored by a fiber optic device and a magnetic field. The magnetic particles will accumulate at the area defined by the magnetic field regardless of whether an analyte is present. However, if an analyte is present that is capable of multivalent interactions with the particles, then the non-magnetic particles containing a specific optical signature will also be colocalized at the site defined by the magnetic field. The magnet may be moved to allow for the particles to sink and float to the surface and mixing may again be started followed by application of a magnetic field again to cause localization. The repeated mixing and magnetic localization processes may be repeated multiple times and may be used to monitor the growth of bacteria. In this way the growth may be monitored nearly continuously and provide evidence that bacteria are alive. The repeated cycling may also provide for higher fidelity data and quantitation. To achieve quantitative measurement of the concentrations of analytes, reference signals may be present and these signals may be integrated into the magnetic particles. It will be important to be able to differentiate the reference signals from the detection signals by choice of wavelengths or lifetimes.

Measurements by the disclosed method may be performed directly in bags that contain samples from which the analytes are extracted. For example, a food sample may be placed in a bag and then subjected to mechanical stimulation by a stomacher. After this treatment the same bag may be interrogated by an optical system. Alternatively, analysis may be performed on samples that have been removed from an initial processing bag and added to other vessels. The samples may be fluid suspensions of analyte that may or may not contain the particles. It is possible that magnetic particles may be added and used to selectively remove analytes that bind to them from the original sample. Such a method has the advantages of enabling analysis in a more controlled medium with known characteristics. It may also allow for bacteria to be removed and placed in a growth media. Repeated measurements of bacteria growth media may provide for a measured growth curve. The initial binding and removal may also be done under conditions that promote the strongest binding of the recognition element to the analyte. The conditions may include the presence of different ions, buffers, surfactants, nutrients, or pH.

An additional advantage of the use of magnetic localization is that the emissive or reflective materials may be localized at the sidewalls of a bag or the vessel containing the sample. In the case that the sample is highly scattering this will avoid transmission through the medium for a higher fidelity measurement. Additionally, this method and focusing optics may avoid signals arising from background emission from the sample.

The method also allows for larger volumes of samples to be analyzed, which may be useful to detect trace levels of an analyte. For example, in the case of bacteria a small number of copies of may be present in a solution and with too small of a sample size, it may be that no bacteria are collected.

### EXAMPLES

The following examples are intended to illustrate certain embodiments of the present invention, but do not exemplify the full scope of the invention.

### Example 1—Preparation of Poly[(9,9-di-n-octylfluorenyl-2,7,diyl)-alt-(benzo[2,1,3]thiadiazol-4,8-diyl)] and (E)-cyclooct-4-en-1-yl poly(ethylene glycol)-poly(styrene) Particles

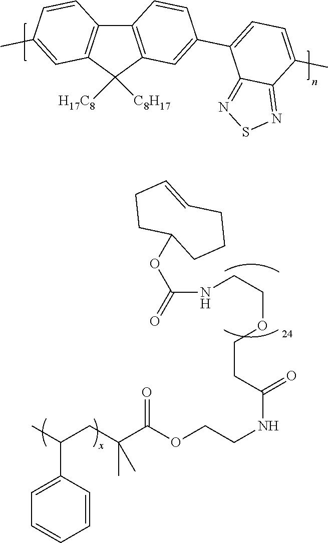

A solution of Poly[(9,9-di-n-octylfluorenyl-2,7,diyl)-alt-(benzo[2,1,3]thiadiazol-4,8-diyl)] (100 μL, 1 mg/mL in tetrahydrofuran (THF)) and (E)-cyclooct-4-en-1-yl poly(ethylene glycol)-poly(styrene) (50 μL, 1 mg/mL in THF) in 5 mL of THF was sonicated for a minute. The mixture was then rapidly pipetted into 10 mL of ultrapure water and sonicated for 2 minutes. The solution was then heated to 85° C. to let the THF evaporate until a volume of about 5-7 mL was reached. 0.5 mL of the solution was incubated with 0.1 mL of LSPS (0.1 mg/mL in 1×PBS) on a RotoTherm Mixer at room temperature overnight. The solution was then dialyzed against PBS 1× through a 20 kDa membrane. Highly fluorescent polymer particles were produced that can be reacted with antibodies or other proteins containing tetrazine groups to produce a functional non-magnetic particle with a signaling entity.

### Example 2—Preparation of Polystyrene Lumogen Red 305 Particles

Using a microfluidic system precision droplets containing 98.4% by weight divinyl-benzene/styrene (4/1), 0.1% by weight (E)-cyclooct-4-en-1-yl poly(ethylene glycol)-poly(styrene), 0.2% by weight Lumogen Red 305, and 1.3% of diphenyl(2,4,6-trimethylbenzoyl)phosphine were dispersed in water containing poly(vinyl alcohol). The droplets were irradiated at 365 nm to give solid non-magnetic luminescent particles suitable for functionalization with recognition elements.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A system for monitoring of a pathogenic analyte, comprising:
a reservoir configured to receive a sample suspected of including the pathogenic analyte, wherein the reservoir is configured to be essentially closed with respect to the pathogenic analyte;
a sterile growth medium formulated to preferentially grow the pathogenic analyte disposed within the reservoir; the growth medium comprising:
a plurality of isolation particles comprising a first moiety capable of binding to the pathogenic analyte, if present; and
a plurality of signaling entities comprising a second moiety capable of binding to the pathogenic analyte, if present.

2. A kit, comprising:
a reservoir configured to receive a pathogenic analyte and a growth medium, wherein the reservoir is configured to be essentially closed with respect to the pathogenic analyte once closed;
a sterile growth medium formulated to preferentially grow the pathogenic analyte disposed within the reservoir; the growth medium comprising a plurality of isolation particles comprising a first moiety capable of binding to the pathogenic analyte.

3. A method for monitoring growth of a pathogenic analyte, the method comprising:
introducing a sample suspected of comprising the pathogenic analyte into a reservoir;
introducing a sterile growth medium formulated to preferentially grow the pathogenic analyte into the reservoir;
closing the reservoir with respect to the pathogenic analyte;
culturing, for a desired period of time, the sample;
mixing the growth medium comprising the sample with a plurality of isolation particles comprising a first moiety capable of binding to the pathogenic analyte, if present;
isolating, via the plurality of isolation particles, the pathogenic analyte; and
determining a property of the pathogenic analyte.

4. A method as in claim 3, further comprising mixing the sterile growth medium comprising the sample with a plurality of signaling entities comprising a second moiety capable of binding to the pathogenic analyte, if present.

5. A method as in any preceding claim, wherein the step of determining the property of the pathogenic analyte comprises exposing the plurality of signaling entities to electromagnetic radiation and detecting, using a detector, a signal produced by the plurality of signaling entities.

6. A method as in any preceding claim, wherein the property is emission from a signaling unit attached to the analyte.

7. A method as in any preceding claim, wherein the plurality of signaling entities is a receptor dye conjugate

8. A method as in any preceding claim, wherein the receptor is an antibody or recognition protein.

9. A method as in any preceding claim, wherein the property is the result of analyte associated reaction that provides a detectable optical change.

10. A method as in any preceding claim, wherein the plurality of signaling entities contains nanoparticles.

11. A method as in any preceding claim, wherein the plurality of signaling entities is an emissive nanoparticle

12. A method as in any preceding claim, wherein the plurality of signaling entities has an excited state lifetime more than 1 microsecond.

13. A method as in any preceding claim, wherein the plurality of signaling entities scatters light.

14. A system for monitoring of a pathogenic analyte, comprising:
a reservoir configured to receive a sample suspected of comprising the pathogenic analyte, wherein the reservoir is configured to be essentially closed with respect to the pathogenic analyte once closed;
a sterile growth medium formulated to preferentially grow the pathogenic analyte disposed within the reservoir,
wherein the pathogenic analyte otherwise requires handling under BSL2 protocol, and
wherein the system does not require a biological safety cabinet or other physical containment equipment to monitor the pathogenic analyte.

15. A method for the detection of a biological analyte through a multivalent interaction that connects magnetic particles and non-magnetic species and allows their localization with an applied magnetic field.

16. A method comprising:
exposing a medium suspected of containing an analyte to a set of magnetic particles and a set of non-magnetic signaling entities, and, if the analyte is present, allowing the analyte to link at least some of the magnetic particles with at least some of the non-magnetic signaling entities;
localizing at least some of the magnetic particles linked to non-magnetic signaling entities via the analyte proximate a source of excitation energy, and localizing at least some of the magnetic particles linked to non-magnetic signaling entities via the analyte proximate a detector;
exciting signaling entities with the source of excitation energy, and determining a signal via the detector, thereby determining a characteristic of the analyte.

17. A method as in any preceding claim, wherein exposing, allowing, localizing, exciting, and determining the signal are conducted in a single medium.

18. A method as in any preceding claim, further comprising providing a set of reference magnetic particles linked to reference signaling entities, localizing at least some of the reference magnetic particles proximate the source of excitation energy and localizing at least some of the reference magnetic particles proximate the detector, exciting reference signaling entities with the source of excitation energy, and determining a reference signal via the detector.

19. A method as in any preceding claim, comprising determining a characteristic of the analyte in part with reference to the reference signal.

20. A composition, comprising a conjugated polymeric species in the form of a signaling entity, optionally, a particulate signaling entity.

21. A system comprising:
a vessel configured to contain a fluid, and including a localization and detection region;
a source of a magnetic field configured to draw magnetic particles proximate the detection region;
a source of excitation energy positioned to expose the detection region to the excitation energy; and
a detector positioned to detect a signal emitted in the detection region.

22. A method as in any previous claim wherein the localization allows for an optical measurement that determines the presence of analyte.

23. A method as in any previous claim wherein the particles are functionalized with a recognition element.

24. A method as in any previous claim wherein the recognition element contains an engineered protein.

25. A method as in any previous claim wherein the recognition element contains an antibody.

26. A method as in any previous claim wherein the recognition element contains a carbohydrate

27. A method as in any previous claim wherein the recognition element contains an oligonucleotide

28. A method as in any previous claim wherein the recognition element contains a synthetic receptor.

29. A method as in any previous claim wherein the magnetic particles contain one or more superparamagnetic particles.

30. A method as in any previous claim wherein the analyte is bacteria.

31. A method as in any previous claim wherein the analyte is a cell.

32. A method as in any previous claim wherein the analyte contains a protein.

33. A method as in any previous claim wherein the analyte is a toxin.

34. A method as in any previous claim wherein the analyte is RNA or DNA.

35. A method as in any previous claim wherein the analyte is a virus.

36. A method as in any previous claim wherein the analyte is an antibody.

37. A method as in any previous claim wherein the magnetic particles contain a signaling entity.

38. A method as in any previous claim wherein the plurality of signaling entities is emissive.

39. A method as in any previous claim wherein one or more of the emissive element is a delayed emission.

40. A method as in any previous claim wherein the plurality of signaling entities comprise a recognition element that is emissive.

41. A method as in any previous claim wherein the plurality of signaling entities comprise an emissive particle containing one or more recognition elements.

42. A method as in any previous claim wherein the plurality of signaling entities comprise an emissive polymer containing one or more recognition elements.

43. A method as in any previous claim wherein the plurality of signaling entities comprise an emissive species containing Eu, Tb, Gd, Au, Au, Ir, Cu, Pd, Pt, Ru, Ag, Zn, or Al.
A method as in any previous claim wherein the non-magnetic species is an emissive polymer is a polyfluorene containing one or more recognition elements.

44. A method as in any previous claim wherein the plurality of signaling entities comprise an emissive polymer capable of transferring energy to a minority chromophore that one or more recognition elements.

45. A method as in any previous claim wherein the measurement can be used to observe bacteria growing.

46. A method as in any previous claim wherein optical signatures allow for measurement of the amount of analyte.

47. A method for determining the presence of an organic analyte by measuring an emission from materials localized by application of a magnetic field.

48. A method as in any previous claim wherein the emission is fluorescence

49. A method as in any previous claim wherein the emission is delayed fluorescence

50. A method as in any previous claim wherein the emission is phosphorescence

51. A method as in any previous claim wherein the localized material is localized on the sidewall of a vessel containing a solution.

52. A method as in any previous claim wherein the localized material is localized by a solution flowing past a magnet

53. A method as in any previous claim wherein the localized material is localized from a solution while a vessel is undergoing physical movement

54. A method as in any previous claim wherein the localized material is localized from a solution while a vessel being stirred

55. A method as in any previous claim wherein the localized material is localized from a solution and the solution is removed

56. A method as in any previous claim wherein the localized material is emissive.
